SEQUENCE LISTING (11 sequences)

SEQ ID NO: 1 - DNA, 5751 bp, Homo sapiens
gagaggttgtaatggaaaaacctagagcaaacgaaaccaatattccttccgagcaatcactaccaggaaaagaggctaccttactagattttggctggagaagtgcatttaaagaagtgtctcttcccatggcgcactgcgtagtaactgcaattgaaggtttttccacaaaaattctccaacaggaacagaatgaaaggtcttcagctgtgagctatgctatgaaccttgtaaatgtccagcaagtttggcaagacagccatatgtttcctgaggaggaacaaccaaagaaaattggaaaattttgttctgacatcatggaaaaacttgacacaatgcttccactggctctggcatgcagagatgattcttttcaggaaattagagcaaacttggtggaggcctgttgtaaagtggcaacagctgtcctgcagagactgcaagagagagccaaggaggttccttccaaagcacccctgaaaaacttgcacacatacctctccacagcggtgtatgtcttccagcatttcaagcgatatgataatttgatgaaggaaatgactaaaaaacccatattcctggtgcttgtccaacgatatcaggaattcatcaacactctacagtttcaggttacgaactactgcgtcagagtttgtgctacaagcattttacaggatgctgagagccaccactgggatgactacaaagctttttatgagggggaaagatgttccttctcgatccagatgtggcattatttctgctggtctcttcattacgatctctggaccattctgcctcctaagttagcccaggagattctagtggaagtgctggagaaatctttgagtctactggcctccagatacgctcgggcccaccccagccgtaaaagaactccacagttaagacttgatgtcacaacaattttgatatgcactgagaacatgttatggtcagtttgtacatctgtacagaaacttttgaatcctcatcagcatacagatgataaaatttttaaaattcacactcattgtaataatctgtttacaacattagtcattttgacttcaccattaacagaattatataagacttttcagcatggcctggatgagtctgcttcagattccttaaaatcattttttaagcaaccattatattgggtttcttgcatatcacatttctacccttctttactcaggactccatcagctggaggactgaaagccgagggtcagttgaaactgctcttatcccagccacgttgtaactggaacttgcttctggaaaccctactgcatcatgatggacttttactgagaatcttgctgaagagctcaaaacgtacctttcaagaacaagtttccgacacagaaaataacctgaaccaaggacccagcttgatggaagccatctttaaaatattgtaccattgcagtttttctccacaaacatttgcaaacgtttttgtgagctacatggaagaagagcaattatgggactttttatataacatcccaggtatgagacatttactgtttgtaaaagaaatgttctatagatacttatatgtacctttaaagatgtttagaagttgtattgatactggttttgctgaatctgttgttggtgaaagtttaccttacattttttcagtataaagccttctgttgggaatcattaattattggttcctgtaaaatttcatatactttgggacaagatgcaagggaggtggaagggaaaagattttctttttttattaattagggaatgttctatcataaaaatttattttaatatttatcacagattatgttttgctattgagcatataagatattagtaagatgtgaaaagtattttgtgattcttgggtgcatttattcagcaaattaatggacaatactattggtcgattttcttcagatgtataaggcatttattattatgaccatagtgtgtctctctacaaggcaagcatatcctgtgattcctttggagagaatatatttttcccatctgtattgttcttcttccttcagctagagagaataataaaggcagtgcaggttgtatatttgaacaggaagttttgatgggttgccccaccctctggcaggcgtaaaaattctcacattgtcttatccttaatattctgcctttaaattttcatatttaatatcttgtgaaaacaaaggatgaaaacttagacaatgatttttattattatcctgctattcaaatagtgatttttatcatttacatttagttttcaacctgtaagctttcttttatcctgctttggaacctgaattttcataaaatatttcctaggctccatataaaagatctaactagttcataatcatggtttcactcctcccagactttttgactatcctccagatattacttgaatatcattctagctgttttacagtcattatgtttcattttctcttggccattatccctataaactagcaatagataactttatgttatgatttccaaagtccaaatctgtcatttaaagtttctgtaataatgatctctttttaaaatgttatctcctagttgccatgcattcccttactgcttcttgcttcccaaaggaagggcatattcctaggtaaaaaggctgaattagaagcttgttgtgtttctgatgtagtctttattgaattgaaagaaaaacgaacttgaaagttctgccaccctctgctgattttagttgtttctggggaagtaagagctggattttctcacaagattataaagaatcagtgaccctcatgcttcaatcactatttatctttcaactattaggttcttgaaccatctttgacactcagcacaaaactatacttaaaataacctagaagatataaatggatttcaggtgctgctagggaaaataaaactgtttcttgctcctaatttgacatctctccatgtatgtagtgagcaaaccctcccctacatggtcagacaagtctctggaaacagacgttatacatattcacagagcctgaattatatgtgaaagctcaaaggggcagaacttcaaatgggtgaaactcccttctcctttatacaagttgttgctccataaacattaaatattctctggaacctgatacctgcagtggttatgagggtctgaagttttctcttttctctcccatttaagtctttgaaaatatctttttttttttctgttagcaatcatggttattttaaaaaccctaataagataatacaaggtactaattcataacgaaattaagccttttactatgacttttgtttaatttggaaacataacattgttacaagtaatacagctgagacatgtaatcataatcccgcaatatttacattttattactaatagatctatgcttcagtatctataattaacctacccagattaactaaagtacattattttaaaacttcactgctgtagtttcctcctgctaaatcactgtctatgatttctgcacagtttacccataaaacatcacagtcttatcattttagggtaaaaggacaaagatgaagtggtatcttcatcttattgttgagtaaacaaaaagcagaaatattaagagagttgcctaagactgtccaccaattaagttggacaagacttagcacaagaaaccagatctcctctctcctaaggcagcagtccccaacatttttggcaccagggaccagttttgtggaagacagtttttccaccaggagggggatggatgaaaccgttccacctcagatcatcaggtattggttagattctcataaggagcaaacaaccaagatcccttgcatgtgcagttcacaataggatttgtgctcctatgagaatctaacatgcttctgatttgacaggaggggaagctcaagcagtaatgctcacccacctgctgctcacctcctgctgtgcggcccagttcctaacaggtcacgaacctgtactggtctgcagtctaggggttggggaccctgtcctaggccatgttatttctcatttttttctattagactaaattttttgagacactacttttatgaacatacgaattcagtaatgaattatacaatctgattttatacttagaattctatctaacacataactctttagactggacataagctagggagggatgacaagaaggaaaatacccacttaagattactacaaaagctataagtacaacaaataattaagtcagtgacctaatgtgggtgtttctaaggccaatgcactctgtaagaaataaatttgctaacaatcattggacacctatgttttcaaagtagatcaagcattattcagttagacaaatgaaaatatcaaagcatacattacctcagctggtttttaagacaatgtcagaacaggcttgtaaatgaggcaaatactgtcaaaaatgcccatctaatatgatagtaatatactcaaaaagtattgtttacttggctcctcgataatggcattgggcttacttttgttttctgaatcagttttcttaactttctcccaacctttgggaaaaaagttttccataacctagcaaaattaagggttgagattcaaagtttccactttggttttctactcctcctcagcctcataaagacctttaagaagtgttgtctagagagcatatatatgctatattgtctgcctgttgccaaaattaatttacagtgccttaccttcctatcagacacagtgattgtctctattacctacataaatacctgcgccatagtataggtagattttaaatgtaagctttcacatattgaaaggctgtctttgttctatgttgtaactaatcatgtattctgagccttttgtcaccagtttgtttgcaatccaccttaaaaatgttttaaaatatatttattgattgaatacaataaaattcactcatttcaagtgaagagttcagtaagtttgaccaatgtataaatctgtgtgacaaccacaacgataaagatatggaacattttatgtcatctccaaaggttctttcattccccctttgccgtccattctcctttcaactctcagccccaggaagacactgacctgctttttgtcgtgatagtttacttttgatattctagaatttcatataaatagaaatctatatattcttttgtgtctagcttcttttgctcagcttaatgtttttgataattttatattatgctttatcattagtttatttctgctcattgctgagcagtatttcactgagtagtactttttttatccacttaccagttgggtggacattttgattatttctagtttttggctacgatgaataaagctgctatgaatactagtgtataagtctttgtgggactacagtcatccctcagtatatgtgaggaattcattccaggaaccattccccttcttaaaaaatccatatactcggccgggcgtggtggctcgcgcctgtggtcccagcactttgggaggccgaagtgggagatcatctgcggtcgagagtttgagaccagcttgaccaacgtggggaggcctcgtttctactgaaaatacaaaattagctgggcatggtggcatgtgcctgcggtcccggctactcgggaggctgaggcaggagagtcgcttgggcccgggaggtggaggttgcggtgggccaagacgccattgcaccccagcctgggcaatgagtggggcttcgtctc

SEQ ID NO: 2 - protein, 553 aa, Homo sapiens
EVVMEKPRANETNIPSEQSLPGKEATLLDFGWRSAFKEVSLPMAHCVVTAIEGFSTKILQQEQNERSSAVSYAMNLVNVQQVWQDSHMFPEEEQPKKIGKFCSDIMEKLDTMLPLALACRDDSFQEIRANLVEACCKVATAVLQRLQERAKEVPSKAPLKNLHTYLSTAVYVFQHFKRYDNLMKEMTKKPIFLVLVQRYQEFINTLQFQVTNYCVRVCATSILQDAESHHWDDYKAFYEGERCSFSIQMWHYFCWSLHYDLWTILPPKLAQEILVEVLEKSLSLLASRYARAHPSRKRTPQLRLDVTTILICTENMLWSVCTSVQKLLNPHQHTDDKIFKIHTHCNNLFTTLVILTSPLTELYKTFQHGLDESASDSLKSFFKQPLYWVSCISHFYPSLLRTPSAGGLKAEGQLKLLLSQPRCNWNLLLETLLHHDGLLLRILLKSSKRTFQEQVSDTENNLNQGPSLMEAIFKILYHCSFSPQTFANVFVSYMEEEQLWDFLYNIPGMRHLLFVKEMFYRYLYVPLKMFRSCIDTGFAESVVGESLPYIFSV

SEQ ID NO: 3 - protein, 228 aa, Rattus norvegicus
TLSPEQLVLTLLEAEPPNVLVSRPSMPFTEASMMMSLTKLADKELVHMIGWAKKIPGFVELSLLDQVRLLESCWMEVLMVGLMWRSIDHPGKLIFAPDLVLDRDEGKCVEGILEIFDMLLATTSRFRELKLQHKEYLCVKAMILLNSSMYPLASANQEAESSRKLTHLLNAVTDALVWVIAKSGISSQQQSVRLANLLMLLSHVRHISNKGMEHLLSMKDLLLEMLNA

SEQ ID NO: 4 - protein, 13 aa, Artificial Sequence
  Description of Artificial Sequence Conserved LBD motif
LXXLXXXDQXXLL

SEQ ID NO: 5 - protein, 13 aa, Artificial Sequence
  Description of Artificial Sequence Conserved LBD motif
IXXIXXXENXXII

SEQ ID NO: 6 - protein, 13 aa, Artificial Sequence
  Description of Artificial Sequence Conserved LBD motif
AXXAXXXXRXXAA

SEQ ID NO: 7 - protein, 13 aa, Artificial Sequence
  Description of Artificial Sequence Conserved LBD motif
VXXVXXXXHXXVV

SEQ ID NO: 8 - protein, 13 aa, Artificial Sequence
  Description of Artificial Sequence Conserved LBD motif
MXXMXXXXKXXMM

SEQ ID NO: 9 - protein, 13 aa, Artificial Sequence
  Description of Artificial Sequence Conserved LBD motif
FXXFXXXXSXXFF

SEQ ID NO: 10 - protein, 13 aa, Artificial Sequence
  Description of Artificial Sequence Conserved LBD motif
YXXYXXXXTXXYY

SEQ ID NO: 11 - protein, 13 aa, Artificial Sequence
  Description of Artificial Sequence Conserved LBD motif
WXXWXXXXXXXWW

(19) United States
(12) Patent Application Publication
(10) Pub. No.: US 2004/0171804 A1
(43) Pub. Date: Sep. 2, 2004

(54) Nuclear hormone receptor ligand binding domain

(76) Inventors: Richard Joseph Fagan, London (GB); Christopher Benjamin Phelps, London (GB)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/469,862

(22) PCT Filed: Mar. 5, 2002
(86) PCT No.: PCT/GB02/00968

(30) Foreign Application Priority Data
Mar. 5, 2001 (GB) 0105401.4

(51) Int. Cl. C07K 14/72

(52) U.S. Cl. 530/350

(57) ABSTRACT

This invention relates to a novel protein, termed BAA74848.1, herein identified as a Nuclear Hormone Receptor Ligand Binding Domain and to the use of this protein and nucleic acid sequence from the encoding gene in the diagnosis, prevention and treatment of disease.

This invention relates to a novel protein, termed BAA74848.1 herein identified as a Nuclear Hormone Receptor Ligand Binding Domain and to the use of this protein and nucleic acid sequence from the encoding gene in the diagnosis, prevention and treatment of disease.

All publications, patents and patent applications cited herein are incorporated in full by reference.

### BACKGROUND

The process of drug discovery is presently undergoing a fundamental revolution as the era of functional genomics comes of age. The term “functional genomics” applies to an approach utilising bioinformatics tools to ascribe function to protein sequences of interest. Such tools are becoming increasingly necessary as the speed of generation of sequence data is rapidly outpacing the ability of research laboratories to assign functions to these protein sequences.

As bioinformatics tools increase in potency and in accuracy, these tools are rapidly replacing the conventional techniques of biochemical characterisation. Indeed, the advanced bioinformatics tools used in identifying the present invention are now capable of outputting results in which a high degree of confidence can be placed.

Various institutions and commercial organisations are examining sequence data as they become available and significant discoveries are being made on an on-going basis. However, there remains a continuing need to identify and characterise further genes and the polypeptides that they encode, as targets for research and for drug discovery.

Recently, a remarkable tool for the evaluation of sequences of unknown function has been developed by the Applicant for the present invention. This tool is a database system, termed the Biopendium search database, that is the subject of co-pending International Patent Application No. PCT/GB01/01105. This database system consists of an integrated data resource created using proprietary technology and containing information generated from an all-by-all comparison of all available protein or nucleic acid sequences.

The aim behind the integration of these sequence data from separate data resources is to combine as much data as possible, relating both to the sequences themselves and to information relevant to each sequence, into one integrated resource. All the available data relating to each sequence, including data on the three-dimensional structure of the encoded protein, if this is available, are integrated together to make best use of the information that is known about each sequence and thus to allow the most educated predictions to be made from comparisons of these sequences. The annotation that is generated in the database and which accompanies each sequence entry imparts a biologically relevant context to the sequence information.

This data resource has made possible the accurate prediction of protein function from sequence alone. Using conventional technology, this is only possible for proteins that exhibit a high degree of sequence identity (above about 20%-30% identity) to other proteins in the same functional family. Accurate predictions are not possible for proteins that exhibit a very low degree of sequence homology to other related proteins of known function.

In the present case, a protein whose sequence is recorded in a publicly available database as KIAA0825 protein (NCBI Genebank nucleotide accession number AB020632 and a Genebank protein accession number BAA74848.1), is implicated as a novel member of the Nuclear Hormone Receptor Ligand Binding Domain family.

### INTRODUCTION TO NUCLEAR HORMONE RECEPTOR LIGAND BINDING DOMAINS

The Nuclear Hormone Receptor gene superfamily (see Table 1) encodes structurally related proteins that regulate the transcription of target genes. These proteins include receptors for steroid and thyroid hormones, vitamins, and other proteins for which no ligands have been found. Nuclear Receptors are composed of two key domains, a DNA-Binding Domain (DBD) and a Ligand Binding Domain (LBD). The DBD directs the receptors to bind specific DNA sequences as monomers, homodimers, or heterodimers. The DBD is a particular type of zinc-finger, found only in Nuclear Receptors. Nuclear Receptors with DBDs can be readily identified at the sequence level by searching for matches to the PROSITE consensus sequence (PS00031).

The Ligand Binding Domain (LBD) binds and responds to the cognate hormone. Ligand binding to the LBD triggers a conformational change which expels a bound “Nuclear Receptor Co-Repressor”. The site previously occupied by the Co-Repressor is then free to recruit a “Nuclear Receptor Co-Activator”. This Ligand-triggered swap of a Co-Repressor for a Co-Activator is the mechanism by which Ligand binding leads to the transcriptional activation of target genes. All ligand binding domains contain a consensus sequence, the “LBD motif” (see Table 2) which mediates Co-Repressor and Co-Activator binding. The LBD is the binding site for all Nuclear Hormone Receptor targeted drugs to date and it is thus desirable to identify novel Ligand Binding Domains since these will be attractive drug targets. Ligand Binding Domains share low sequence identity (˜15%) but have very similar structures and so present ideal targets for a structure-based relationship tool such as Genome Threader.

Many protein sequences have already been annotated in the public domain as Nuclear Hormone Receptors by their possession of DBDs using basic search tools like PROSITE, and their LBDs inferred on the basis of this. Because of this it is anticipated that any novel LBDs identified by Genome Threader which are not annotated as nuclear receptors will lack the DBD entirely. A precedent for a protein which has an LBD but lacks a DBD is provided by DAXI. Thus we annotate these DBD-less hits not as “Nuclear Hormone Receptors” but rather as containing a “Nuclear Hormone Receptor Ligand Binding Domain”.

TABLE 1
Nuclear hormone Receptor Superfamily
Family: Steroid Hormone Receptors

| | |
|---|---|
| Subfamilies | Glucocorticoid Receptors |
| | Progesterone Receptors |
| | Androgen Receptors |
| | Estrogen Receptors |

Family: Thyroid Hormone Receptor-like Factors

| | |
|---|---|
| Subfamilies | Retinoic Acid Receptors (RARs) |
| | Retinoid X Receptors (RXRs) |
| | Thyroid Hormone Receptors |
| | Vitamin D Receptor |
| | NGFI-B |
| | FTZ-F1 |
| | Peroxisome Proliferator Activated Receptors (PPARs) |
| | Ecdysone Receptors |
| | Retinoid Orphan Receptors (RORs) |
| | Tailess/COUP |
| | HNF-4 |
| | CF1 |
| | Knirps |

Family: DAX1

| | |
|---|---|
| Subfamilies | DAX1 |

TABLE 2
The “LBD motif”. Numbers along the top row refer to residue position
within the motif. Letters refer to amino acids by the 1-letter code.
Letters within one column are all acceptable for that position within
the motif. For example L, I, A, V, M, F, Y or W can occupy the first
position of the “LBD motif”. Note that there is observed variation in
the number of residues found between position 4 and 8, and position 9
and 12. The “LBD motif” was constructed by aligning 681 sequences
of Nuclear Hormone Receptor Ligand Binding Domains, and
identifying conserved patterns of residues.

| | | | | | | | | | | | | |
|---|---|---|---|---|---|---|---|---|---|---|---|---|
| 1 | 2 | 3 | 4 | 5 | 6 | 7 | 8 | 9 | 10 | 11 | 12 | 13 |

| | | | | | | | | |
|---|---|---|---|---|---|---|---|---|
| L | Any 2 | L | Any 3 residues | D | Q | Any 2 | L | L |
| I | residues | I | (or 2 residues | E | N | residues | I | I |
| A | | A | or 4 residues) | | R | (or 1 or 3 | A | A |
| V | | V | | | H | residues) | V | V |
| M | | M | | | K | | M | M |
| F | | F | | | S | | F | F |
| Y | | Y | | | T | | Y | Y |
| W | | W | | | | | W | W |

### NUCLEAR HORMONE RECEPTORS AND DISEASE

Nuclear Hormone Receptors have been shown to play a role in diverse physiological functions, many of which can play a role in disease processes (see Table 3).

TABLE 3
Nuclear Hormone Receptors and disease.

| Nuclear Hormone | |
|---|---|
| Receptor | Disease |
| Androgen Receptor | Androgen Insensitivity Syndrome (Lubahn et al. 1989 |
| | Proc. Natl. Acad. Sci. USA 86, 9534-9538). |
| | Reifenstein syndrome (Wooster et al. 1992 Nat. Genet. 2, |
| | 132-134). |
| | X-linked recessive spinal and bulbar muscular atrophy |
| | (MacLean et al. 1995 Mol. Cell. Endocrinol. 112, 133-141). |
| | Male breast cancer ((Wooster et al. 1992 Nat. Genet. 2, |
| | 132-134). |
| Glucocorticoid Receptor | Nelson's syndrome (Karl et al. 1996 J. Clin. Endocrinol. |
| | Metab. 81, 124-129). |
| | Glucocorticoid resistant acute T-cell leukemia (Hala et al. |
| | 1996 Int. J. Cancer 68, 663-668). |
| Mineralocorticoid | Pseudohypoaldosteronism (Chung et al. 1995 J. Clin. |
| Receptor | Endocrinol. Metab. 80, 3341-3345). |
| Estrogen Receptor alpha | ER alpha expression is elevated in a subset of human |
| | breast cancers. The application of Tamoxifen is the major |
| | therapy to prevent breast tumour progression. |
| | Unfortunately 35% of ER alpha positive breast cancers are |
| | Tamoxifen resistant (Petrangeli et al. 1994 J. Steroid |
| | Biochem. Mol. Biol. 49, 327-331). |
| Vitamin D3 Receptor | Mutations in the Vitamin D3 receptor produce a |
| | hereditary disorder similar in phenotype to Vitamin D3 |
| | deficiency (Rickets) (Hughes et al. 1988 Science 242, |
| | 1702-1725). |
| Retinoic Acid Receptor | Acute Myeloid Leukemia (Lavau and Dejean 1994 |
| alpha | Leukemia 8, 9-15). |
| Thyroid Hormone | “Generalised Resistance to Thyroid Hormones” (GRTH) |
| Receptor beta | (Refetoff 1994 Thyroid 4, 345-349). |
| DAX1 | X-linked Adrenal Hypoplasia Congenita (AHC) and |
| | Hypogonadism (Ito et al. 1997 Mol. Cell. Biol. 17, 1476-1483). |

Alteration of Nuclear Hormone Receptors by ligands which bind to their LBD thus provides a means to alter the disease phenotype. There is thus a great need for the identification of novel Nuclear Hormone Receptor Ligand Binding Domains, as these proteins may play a role in the diseases identified above, as well as in other disease states. The identification of novel Nuclear Hormone Receptor Ligand Binding Domains is thus highly relevant for the treatment and diagnosis of disease, particularly those identified in Table 3.

### THE INVENTION

The invention is based on the discovery that the BAA74848.1 protein functions as a Nuclear Hormone Receptor Ligand Binding Domain.

For the BAA74848.1 protein, it has been found that a region including residues 114-301 of this protein sequence adopts an equivalent fold to residues 16 (Pro232) to 192 (Val408) of the Rat Oestrogen Receptor beta (PDB code 1QKN:A). Rat Oestrogen Receptor beta is known to function as a Nuclear Hormone Receptor Ligand Binding Domain. Furthermore, the “LBD motif” residues PHE274, LEU277, ASP281, GLN282, LEU285 and LEU286 of the Rat Oestrogen Receptor beta are conserved as PHE173, PHE176, ASP180, ASN181, LEU182 and MET 183 in BAA74848.1, respectively. This relationship is not just to Rat Oestrogen Receptor beta, but rather to the Nuclear Hormone Receptor Ligand Binding Domain family as a whole. Thus, by reference to the Genome Threader™ alignment of BAA74848.1 with the Rat Oestrogen Receptor beta (1QKN:A) PHE173, PHE176, ASP180, ASN181, Leu182 and Met 183 of BAA74848.1 are predicted to form the “LBD motif” residues.

The combination of equivalent fold and conservation of “LBD motif” residues allows the functional annotation of this region of BAA74848.1, and therefore proteins that include this region, as possessing Nuclear Hormone Receptor Ligand Binding Domain activity. In a first aspect, the invention provides a polypeptide, which polypeptide:

(i) comprises the amino acid sequence as recited in SEQ ID NO:2;

(ii) is a fragment thereof having Nuclear Hormone Receptor Ligand Binding Domain activity or having an antigenic determinant in common with the polypeptides of (i); or

(iii) is a functional equivalent of (i) or (ii).

Preferably, the polypeptide:

(i) consists of the amino acid sequence as recited in SEQ ID NO:2;

(ii) is a fragment thereof having Nuclear Hormone Receptor Ligand Binding Domain activity or having an antigenic determinant in common with the polypeptides of (i); or

(iii) is a functional equivalent of (i) or (ii).

The polypeptide having the sequence recited in SEQ ID NO:2 is referred to hereafter as “the LBDS2 polypeptide”.

According to this aspect of the invention, a preferred polypeptide fragment according to part ii) above includes the region of the LBDS2 polypeptide that is predicted as that responsible for Nuclear Hormone Receptor Ligand Binding Domain activity (hereafter, the “LBDS2 Nuclear Hormone Receptor Ligand Binding Domain region”), or is a variant thereof that possesses the “LBD motif” (PHE173, PHE176, ASP180, ASN181, Leu182 and Met 183, or equivalent residues). As defined herein, the LBDS2 Nuclear Hormone Receptor Ligand Binding Domain region is considered to extend residue 114 and residue 301 of the LBDS2 polypeptide sequence.

This aspect of the invention also includes fusion proteins that incorporate polypeptide fragments and variants of these polypeptide fragments as defined above, provided that said fusion proteins possess activity as a Nuclear Hormone Receptor Ligand Binding Domain.

In a second aspect, the invention provides a purified nucleic acid molecule that encodes a polypeptide of the first aspect of the invention. Preferably, the purified nucleic acid molecule has the nucleic acid sequence as recited in SEQ ID NO:1 (encoding the LBDS2 polypeptide), or is a redundant equivalent or fragment of this sequence. A preferred nucleic acid fragment is one that encodes a polypeptide fragment according to part ii) above, preferably a polypeptide fragment that includes the LBDS2 Nuclear Hormone Receptor Ligand Binding Domain region, or that encodes a variant of these fragments as this term is defined above.

In a third aspect, the invention provides a purified nucleic acid molecule which hybridizes under high stringency conditions with a nucleic acid molecule of the second aspect of the invention.

In a fourth aspect, the invention provides a vector, such as an expression vector, that contains a nucleic acid molecule of the second or third aspect of the invention.

In a fifth aspect, the invention provides a host cell transformed with a vector of the fourth aspect of the invention.

In a sixth aspect, the invention provides a ligand which binds specifically to, and which preferably inhibits the Nuclear Hormone Receptor Ligand Binding Domain activity of, a polypeptide of the first aspect of the invention.

In a seventh aspect, the invention provides a compound that is effective to alter the expression of a natural gene which encodes a polypeptide of the first aspect of the invention or to regulate the activity of a polypeptide of the first aspect of the invention.

A compound of the seventh aspect of the invention may either increase (agonise) or decrease (antagonise) the level of expression of the gene or the activity of the polypeptide. Importantly, the identification of the function of the region defined herein as the LBDS2 Nuclear Hormone Receptor Ligand Binding Domain region of the LBDS2 polypeptide, respectively, allows for the design of screening methods capable of identifying compounds that are effective in the treatment and/or diagnosis of diseases in which Nuclear Hormone Receptor Ligand Binding Domains are implicated.

In an eighth aspect, the invention provides a polypeptide of the first aspect of the invention, or a nucleic acid molecule of the second or third aspect of the invention, or a vector of the fourth aspect of the invention, or a ligand of the fifth aspect of the invention, or a compound of the sixth aspect of the invention, for use in therapy or diagnosis. These molecules may also be used in the manufacture of a medicament for the treatment of cell proliferative disorders, including neoplasm, cancer and myeloproliferative disorders and angiogenesis, metabolic disorders, including obesity, osteoporosis, non-insulin dependent diabetes, lipid metabolism disorder, hyperthyroidism, hyperparathyroidism, hypercalcemia, hypercholestrolemia, hyperlipidemia, cardiovascular disorders including, hypertension, athersclerosis, hyperinsulinemia, heart arrhythmia, renal disorders, including glomerulonephritis, renovascular hypertension, skin disorders including dermatological disease, psoriasis, acne, aging, eczema, wound healing, inflammation, including inflammatory bowel disease, respiratory tract inflammation, emphysema, asthma, immune disorder, autoimmune disease, multiple sclerosis, nervous system disorders, including anxiety, depression, neurodegenerative disease, Alzheimer's disease Parkinson's disease, brain injury, stroke and pain, infection, including virus infection, and other conditions in which nuclear hormones are implicated.

In a ninth aspect, the invention provides a method of diagnosing a disease in a patient, comprising assessing the level of expression of a natural gene encoding a polypeptide of the first aspect of the invention or the activity of a polypeptide of the first aspect of the invention in tissue from said patient and comparing said level of expression or activity to a control level, wherein a level that is different to said control level is indicative of disease. Such a method will preferably be carried out in vitro. Similar methods may be used for monitoring the therapeutic treatment of disease in a patient, wherein altering the level of expression or activity of a polypeptide or nucleic acid molecule over the period of time towards a control level is indicative of regression of disease.

A preferred method for detecting polypeptides of the first aspect of the invention comprises the steps of: (a) contacting a ligand, such as an antibody, of the sixth aspect of the invention with a biological sample under conditions suitable for the formation of a ligand-polypeptide complex; and (b) detecting said complex.

A number of different such methods according to the ninth aspect of the invention exist, as the skilled reader will be aware, such as methods of nucleic acid hybridization with short probes, point mutation analysis, polymerase chain reaction (PCR) amplification and methods using antibodies to detect aberrant protein levels. Similar methods may be used on a short or long term basis to allow therapeutic treatment of a disease to be monitored in a patient. The invention also provides kits that are useful in these methods for diagnosing disease.

In a tenth aspect, the invention provides for the use of a polypeptide of the first aspect of the invention as a Nuclear Hormone Receptor Ligand Binding Domain. The invention also provides for the use of a nucleic acid molecule according to the second or third aspects of the invention to express a protein that possesses Nuclear Hormone Receptor Ligand Binding Domain activity. The invention also provides a method for effecting Nuclear Hormone Receptor Ligand Binding Domain activity, said method utilising a polypeptide of the first aspect of the invention.

In an eleventh aspect, the invention provides a pharmaceutical composition comprising a polypeptide of the first aspect of the invention, or a nucleic acid molecule of the second or third aspect of the invention, or a vector of the fourth aspect of the invention, or a ligand of the sixth aspect of the invention, or a compound of the seventh aspect of the invention, in conjunction with a pharmaceutically-acceptable carrier.

In a twelfth aspect, the present invention provides a polypeptide of the first aspect of the invention, or a nucleic acid molecule of the second or third aspect of the invention, or a vector of the fourth aspect of the invention, or a ligand of the sixth aspect of the invention, or a compound of the seventh aspect of the invention, for use in the manufacture of a medicament for the diagnosis or treatment of a disease, such as cell proliferative disorders, including neoplasm, cancer and myeloproliferative disorders and angiogenesis, metabolic disorders, including obesity, osteoporosis, non-insulin dependent diabetes, lipid metabolism disorder, hyperthyroidism, hyperparathyroidism, hypercalcemia, hypercholestrolemia, hyperlipidemia, cardiovascular disorders including, hypertension, athersclerosis, hyperinsulinemia, heart arrhythmia, renal disorders, including glomerulonephritis, renovascular hypertension, skin disorders including dermatological disease, psoriasis, acne, aging, eczema, wound healing, inflammation, including inflammatory bowel disease, respiratory tract inflammation, emphysema, asthma, immune disorder, autoimmune disease, multiple sclerosis, nervous system disorders, including anxiety, depression, neurodegenerative disease, Alzheimer's disease Parkinson's disease, brain injury, stroke and pain, infection, including virus infection, and other conditions in which nuclear hormones are implicated.

In a thirteenth aspect, the invention provides a method of treating a disease in a patient comprising administering to the patient a polypeptide of the first aspect of the invention, or a nucleic acid molecule of the second or third aspect of the invention, or a vector of the fourth aspect of the invention, or a ligand of the sixth aspect of the invention, or a compound of the seventh aspect of the invention.

For diseases in which the expression of a natural gene encoding a polypeptide of the first aspect of the invention, or in which the activity of a polypeptide of the first aspect of the invention, is lower in a diseased patient when compared to the level of expression or activity in a healthy patient, the polypeptide, nucleic acid molecule, ligand or compound administered to the patient should be an agonist. Conversely, for diseases in which the expression of the natural gene or activity of the polypeptide is higher in a diseased patient when compared to the level of expression or activity in a healthy patient, the polypeptide, nucleic acid molecule, ligand or compound administered to the patient should be an antagonist. Examples of such antagonists include antisense nucleic acid molecules, ribozymes and ligands, such as antibodies.

In a fourteenth aspect, the invention provides transgenic or knockout non-human animals that have been transformed to express higher, lower or absent levels of a polypeptide of the first aspect of the invention. Such transgenic animals are very useful models for the study of disease and may also be using in screening regimes for the identification of compounds that are effective in the treatment or diagnosis of such a disease.

A summary of standard techniques and procedures which may be employed in order to utilise the invention is given below. It will be understood that this invention is not limited to the particular methodology, protocols, cell lines, vectors and reagents described. It is also to be understood that the terminology used herein is for the purpose of describing particular embodiments only and it is not intended that this terminology should limit the scope of the present invention. The extent of the invention is limited only by the terms of the appended claims.

Standard abbreviations for nucleotides and amino acids are used in this specification.

The practice of the present invention will employ, unless otherwise indicated, conventional techniques of molecular biology, microbiology, recombinant DNA technology and immunology, which are within the skill of those working in the art.

Such techniques are explained fully in the literature. Examples of particularly suitable texts for consultation include the following: Sambrook Molecular Cloning; A Laboratory Manual, Second Edition (1989); DNA Cloning, Volumes I and II (D. N. Glover ed. 1985); Oligonucleotide Synthesis (M. J. Gait ed. 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Immunochemical Methods in Cell and Molecular Biology (Mayer and Walker, eds. 1987, Academic Press, London); Scopes, (1987) Protein Purification: Principles and Practice, Second Edition (Springer Verlag, N.Y.); and Handbook of Experimental Immunology, Volumes I-IV (D.M. Weir and C. C. Blackwell eds. 1986).

As used herein, the term “polypeptide” includes any peptide or protein comprising two or more amino acids joined to each other by peptide bonds or modified peptide bonds, i.e. peptide isosteres. This term refers both to short chains (peptides and oligopeptides) and to longer chains (proteins).

The polypeptide of the present invention may be in the form of a mature protein or may be a pre-, pro- or prepro-protein that can be activated by cleavage of the pre-, pro- or prepro-portion to produce an active mature polypeptide. In such polypeptides, the pre-, pro- or prepro-sequence may be a leader or secretory sequence or may be a sequence that is employed for purification of the mature polypeptide sequence.

The polypeptide of the first aspect of the invention may form part of a fusion protein. For example, it is often advantageous to include one or more additional amino acid sequences which may contain secretory or leader sequences, pro-sequences, sequences which aid in purification, or sequences that confer higher protein stability, for example during recombinant production. Alternatively or additionally, the mature polypeptide may be fused with another compound, such as a compound to increase the half-life of the polypeptide (for example, polyethylene glycol).

Polypeptides may contain amino acids other than the 20 gene-encoded amino acids, modified either by natural processes, such as by post-translational processing or by chemical modification techniques which are well known in the art. Among the known modifications which may commonly be present in polypeptides of the present invention are glycosylation, lipid attachment, sulphation, gamma-carboxylation, for instance of glutamic acid residues, hydroxylation and ADP-ribosylation. Other potential modifications include acetylation, acylation, amidation, covalent attachment of flavin, covalent attachment of a haeme moiety, covalent attachment of a nucleotide or nucleotide derivative, covalent attachment of a lipid derivative, covalent attachment of phosphatidylinositol, cross-linking, cyclization, disulphide bond formation, demethylation, formation of covalent cross-links, formation of cysteine, formation of pyroglutamate, formylation, GPI anchor formation, iodination, methylation, myristoylation, oxidation, proteolytic processing, phosphorylation, prenylation, racemization, selenoylation, transfer-RNA mediated addition of amino acids to proteins such as arginylation, and ubiquitination.

Modifications can occur anywhere in a polypeptide, including the peptide backbone, the amino acid side-chains and the amino or carboxyl termini. In fact, blockage of the amino or carboxyl terminus in a polypeptide, or both, by a covalent modification is common in naturally-occurring and synthetic polypeptides and such modifications may be present in polypeptides of the present invention.

The modifications that occur in a polypeptide often will be a function of how the polypeptide is made. For polypeptides that are made recombinantly, the nature and extent of the modifications in large part will be determined by the post-translational modification capacity of the particular host cell and the modification signals that are present in the amino acid sequence of the polypeptide in question. For instance, glycosylation patterns vary between different types of host cell.

The polypeptides of the present invention can be prepared in any suitable manner. Such polypeptides include isolated naturally-occurring polypeptides (for example purified from cell culture), recombinantly-produced polypeptides (including fusion proteins), synthetically-produced polypeptides or polypeptides that are produced by a combination of these methods.

The functionally-equivalent polypeptides of the first aspect of the invention may be polypeptides that are homologous to the LBDS2 polypeptide. Two polypeptides are said to be “homologous”, as the term is used herein, if the sequence of one of the polypeptides has a high enough degree of identity or similarity to the sequence of the other polypeptide. “Identity” indicates that at any particular position in the aligned sequences, the amino acid residue is identical between the sequences. “Similarity” indicates that, at any particular position in the aligned sequences, the amino acid residue is of a similar type between the sequences. Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

Homologous polypeptides therefore include natural biological variants (for example, allelic variants or geographical variations within the species from which the polypeptides are derived) and mutants (such as mutants containing amino acid substitutions, insertions or deletions) of the LBDS2 polypeptide. Such mutants may include polypeptides in which one or more of the amino acid residues are substituted with a conserved or non-conserved amino acid residue (preferably a conserved amino acid residue) and such substituted amino acid residue may or may not be one encoded by the genetic code. Typical such substitutions are among Ala, Val, Leu and Ile; among Ser and Thr; among the acidic residues Asp and Glu; among Asn and Gln; among the basic residues Lys and Arg; or among the aromatic residues Phe and Tyr. Particularly preferred are variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the protein. Also especially preferred in this regard are conservative substitutions.

Such mutants also include polypeptides in which one or more of the amino acid residues include a substituent group.

Typically, greater than 80% identity between two polypeptides (preferably, over a specified region) is considered to be an indication of functional equivalence. Preferably, functionally equivalent polypeptides of the first aspect of the invention have a degree of sequence identity with the LBDS2 polypeptide, or with active fragments thereof, of greater than 80%. More preferred polypeptides have degrees of identity of greater than 85%, 90%, 95%, 98% or 99%, respectively with the LBDS2 polypeptide, or with active fragments thereof.

Percentage identity, as referred to herein, is as determined using BLAST version 2.1.3 using the default parameters specified by the NCBI (the National Center for Biotechnology Information; http://www.ncbi.nlm.nih.gov/) [Blosum 62 matrix; gap open penalty=11 and gap extension penalty=1].

In the present case, preferred active fragments of the LBDS2 polypeptide are those that include the LBDS2 Nuclear Hormone Receptor Ligand Binding Domain region and which possess the “LBD motif” of residues PHE173, PHE176, ASP180, ASN181, Leu182 and Met 183, or equivalent residues. By “equivalent residues” is meant residues that are equivalent to the “LBD motif” residues, provided that the Nuclear Hormone Receptor Ligand Binding Domain region retains activity as a Nuclear Hormone Receptor Ligand Binding Domain. For example PHE173 may be replaced by LEU, ILE, ALA, VAL, MET, TYR or TRP. For example PHE176 may be replaced by LEU, ILE, ALA, VAL, MET, TYR or TRP. For example ASP180 may be replaced by GLU. For example ASN181 may be replaced by GLN, ARG, HIS, LYS, SER or THR. For example LEU182 may be replaced by ILE, ALA, VAL, MET, PHE, TYR, or TRP. For example MET183 may be replaced by LEU, ILE, ALA, VAL, PHE, TYR, or TRP. Accordingly, this aspect of the invention includes polypeptides that have degrees of identity of greater than 80%, preferably, greater than 85%, 90%, 95%, 98% or 99%, respectively, with the Nuclear Hormone Receptor Ligand Binding Domain region of the LBDS2 polypeptide and which possess the “LBD motif” of PHE173, PHE176, ASP180, ASN181, Leu182 and Met 183, or equivalent residues. As discussed above, the LBDS2 Nuclear Hormone Receptor Ligand Binding Domain region is considered to extend between residue 114 and residue 301 of the LBDS2 polypeptide sequence.

The functionally-equivalent polypeptides of the first aspect of the invention may also be polypeptides which have been identified using one or more techniques of structural alignment. For example, the Inpharmatica Genome Threader™ technology that forms one aspect of the search tools used to generate the Biopendium search database may be used (see co-pending International patent application PCT/GB01/01105) to identify polypeptides of presently-unknown function which, while having low sequence identity as compared to the LBDS2 polypeptide, are predicted to have Nuclear Hormone Receptor Ligand Binding Domain activity, by virtue of sharing significant structural homology with the LBDS2 polypeptide sequence.

By “significant structural homology” is meant that the Inpharmatica Genome Threader™ predicts two proteins, or protein regions, to share structural homology with a certainty of at least 10% more preferably, at least 20%, 30%, 40%, 50%, 60%, 70%, 80%, 90% and above. The certainty value of the Inpharmatica Genome Threader™ is calculated as follows. A set of comparisons was initially performed using the Inpharmatica Genome Threader™ exclusively using sequences of known structure. Some of the comparisons were between proteins that were known to be related (on the basis of structure). A neural network was then trained on the basis that it needed to best distinguish between the known relationships and known not-relationships taken from the CATH structure classification (www.biochem.ucl.ac.uk/bsm/cath). This resulted in a neural network score between 0 and 1. However, again as the number of proteins that are related and the number that are unrelated were known, it was possible to partition the neural network results into packets and calculate empirically the percentage of the results that were correct. In this manner, any genuine prediction in the Biopendium search database has an attached neural network score and the percentage confidence is a reflection of how successful the Inpharmatica Genome Threader™ was in the training/testing set.

Structural homologues of LBDS2 should share structural homology with the LBDS2 Nuclear Hormone Receptor Ligand Binding Domain region and possess the “LBD motif” residues PHE173, PHE176, ASP180, ASN181, Leu182 and Met 183, or equivalent residues. Such structural homologues are predicted to have Nuclear Hormone Receptor Ligand Binding Domain activity by virtue of sharing significant structural homology with this polypeptide sequence and possessing the “LBD motif” residues.

The polypeptides of the first aspect of the invention also include fragments of the LBDS2 polypeptide, functional equivalents of the fragments of the LBDS2 polypeptide, and fragments of the functional equivalents of the LBDS2 polypeptides, provided that those functional equivalents and fragments retain Nuclear Hormone Receptor Ligand Binding Domain activity or have an antigenic determinant in common with the LBDS2 polypeptide.

As used herein, the term “fragment” refers to a polypeptide having an amino acid sequence that is the same as part, but not all, of the amino acid sequence of the LBDS2 polypeptides or one of its functional equivalents. The fragments should comprise at least n consecutive amino acids from the sequence and, depending on the particular sequence, n preferably is 7 or more (for example, 8, 10, 12, 14, 16, 18, 20 or more). Small fragments may form an antigenic determinant.

Preferred polypeptide fragments according to this aspect of the invention are fragments that include a region defined herein as the LBDS2 Nuclear Hormone Receptor Ligand Binding Domain region of the LBDS2 polypeptides, respectively. These regions are the regions that have been annotated as Nuclear Hormone Receptor Ligand Binding Domain.

For the LBDS2 polypeptide, this region is considered to extend between residue 114 and residue 301.

Variants of this fragment are included as embodiments of this aspect of the invention, provided that these variants possess activity as a Nuclear Hormone Receptor Ligand Binding Domain.

In one respect, the term “variant” is meant to include extended or truncated versions of this polypeptide fragment.

For extended variants, it is considered highly likely that the Nuclear Hormone Receptor Ligand Binding Domain region of the LBDS2 polypeptide will fold correcdy and show Nuclear Hormone Receptor Ligand Binding Domain activity if additional residues C terminal and/or N terminal of these boundaries in the LBDS2 polypeptide sequence are included in the polypeptide fragment. For example, an additional 5, 10, 20, 30, 40 or even 50 or more amino acid residues from the LBDS2 polypeptide sequence, or from a homologous sequence, may be included at either or both the C terminal and/or N terminal of the boundaries of the Nuclear Hormone Receptor Ligand Binding Domain regions of the LBDS2 polypeptide, without prejudicing the ability of the polypeptide fragment to fold correctly and exhibit Nuclear Hormone Receptor Ligand Binding Domain activity.

For truncated variants of the LBDS2 polypeptide, one or more amino acid residues may be deleted at either or both the C terminus or the N terminus of the Nuclear Hormone Receptor Ligand Binding Domain region of the LBDS2 polypeptide, although the “LBD motif” residues (PHE173, PHE176, ASP180, ASN181, Leu182 and Met 183), or equivalent residues should be maintained intact; deletions should not extend so far into the polypeptide sequence that any of these residues are deleted.

In a second respect, the term “variant” includes homologues of the polypeptide fragments described above, that possess significant sequence homology with the Nuclear Hormone Receptor Ligand Binding Domain region of the LBDS2 polypeptide and which possess the “LBD motif” residues (PHE173, PHE176, ASP180, ASN181, Leu182 and Met 183), or equivalent residues, provided that said variants retain activity as an Nuclear Hormone Receptor Ligand Binding Domain.

Homologues include those polypeptide molecules that possess greater than 30% identity with the LBDS2 Nuclear Hormone Receptor Ligand Binding Domain regions, of the LBDS2 polypeptides, respectively. Percentage identity is as determined using BLAST version 2.1.3 using the default parameters specified by the NCBI (the National Center for Biotechnology Information; http://www.ncbi.nlm.nih.gov/) [Blosum 62 matrix; gap open penalty=11 and gap extension penalty=1]. Preferably, variant homologues of polypeptide fragments of this aspect of the invention have a degree of sequence identity with the LBDS2 Nuclear Hormone Receptor Ligand Binding Domain regions, of the LBDS2 polypeptides, respectively, of greater than 80%. More preferred variant polypeptides have degrees of identity of greater than 85%, 90%, 95%, 98% or 99%, respectively with the LBDS2 Nuclear Hormone Receptor Ligand Binding Domain regions of the LBDS2, polypeptides, provided that said variants retain activity as a Nuclear Hormone Receptor Ligand Binding Domain. Variant polypeptides also include homologues of the truncated forms of the polypeptide fragments discussed above, provided that said variants retain activity as a Nuclear Hormone Receptor Ligand Binding Domain.

The polypeptide fragments of the first aspect of the invention may be polypeptide fragments that exhibit significant structural homology with the structure of the polypeptide fragment defined by the LBDS2 Nuclear Hormone Receptor Ligand Binding Domain regions, of the LBDS2 polypeptide sequence, for example, as identified by the Inpharmatica Genome Threader™. Accordingly, polypeptide fragments that are structural homologues of the polypeptide fragments defined by the LBDS2 Nuclear Hormone Receptor Ligand Binding Domain regions of the LBDS2 polypeptide sequence should adopt the same fold as that adopted by this polypeptide fragment, as this fold is defined above.

Structural homologues of the polypeptide fragment defined by the LBDS2 Nuclear Hormone Receptor Ligand Binding Domain region should also retain the “LBD motif” residues PHE173, PHE176, ASP180, ASN181, LEU182 and MET 183, or equivalent residues.

Such fragments may be “free-standing”, i.e. not part of or fused to other amino acids or polypeptides, or they may be comprised within a larger polypeptide of which they form a part or region. When comprised within a larger polypeptide, the fragment of the invention most preferably forms a single continuous region. For instance, certain preferred embodiments relate to a fragment having a pre- and/or pro-polypeptide region fused to the amino terminus of the fragment and/or an additional region fused to the carboxyl terminus of the fragment. However, several fragments may be comprised within a single larger polypeptide.

The polypeptides of the present invention or their immunogenic fragments (comprising at least one antigenic determinant) can be used to generate ligands, such as polyclonal or monoclonal antibodies, that are immunospecific for the polypeptides. Such antibodies may be employed to isolate or to identify clones expressing the polypeptides of the invention or to purify the polypeptides by affinity chromatography. The antibodies may also be employed as diagnostic or therapeutic aids, amongst other applications, as will be apparent to the skilled reader.

The term “immunospecific” means that the antibodies have substantially greater affinity for the polypeptides of the invention than their affinity for other related polypeptides in the prior art. As used herein, the term “antibody” refers to intact molecules as well as to fragments thereof, such as Fab, F(ab′)2 and Fv, which are capable of binding to the antigenic determinant in question. Such antibodies thus bind to the polypeptides of the first aspect of the invention.

If polyclonal antibodies are desired, a selected mammal, such as a mouse, rabbit, goat or horse, may be immunised with a polypeptide of the first aspect of the invention. The polypeptide used to immunise the animal can be derived by recombinant DNA technology or can be synthesized chemically. If desired, the polypeptide can be conjugated to a carrier protein. Commonly used carriers to which the polypeptides may be chemically coupled include bovine serum albumin, thyroglobulin and keyhole limpet haemocyanin. The coupled polypeptide is then used to immunise the animal. Serum from the immunised animal is collected and treated according to known procedures, for example by immunoaffinity chromatography.

Monoclonal antibodies to the polypeptides of the first aspect of the invention can also be readily produced by one skilled in the art. The general methodology for making monoclonal antibodies using hybridoma technology is well known (see, for example, Kohler, G. and Milstein, C., Nature 256: 495497 (1975); Kozbor et al., Immunology Today 4: 72 (1983); Cole et al., 77-96 in Monoclonal Antibodies and Cancer Therapy, Alan R. Liss, Inc. (1985).

Panels of monoclonal antibodies produced against the polypeptides of the first aspect of the invention can be screened for various properties, i.e., for isotype, epitope, affinity, etc. Monoclonal antibodies are particularly useful in purification of the individual polypeptides against which they are directed. Alternatively, genes encoding the monoclonal antibodies of interest may be isolated from hybridomas, for instance by PCR techniques known in the art, and cloned and expressed in appropriate vectors.

Chimeric antibodies, in which non-human variable regions are joined or fused to human constant regions (see, for example, Liu et al., Proc. Natl. Acad. Sci. USA, 84, 3439 (1987)), may also be of use.

The antibody may be modified to make it less immunogenic in an individual, for example by humanisation (see Jones et al., Nature, 321, 522 (1986); Verhoeyen et al., Science, 239: 1534 (1988); Kabat et al., J. Immunol., 147: 1709 (1991); Queen et al., Proc. Natl Acad. Sci. USA, 86, 10029 (1989); Gorman et al., Proc. Natl Acad. Sci. USA, 88: 34181 (1991); and Hodgson et al., Bio/Technology 9: 421 (1991)). The term “humanised antibody”, as used herein, refers to antibody molecules in which the CDR amino acids and selected other amino acids in the variable domains of the heavy and/or light chains of a non-human donor antibody have been substituted in place of the equivalent amino acids in a human antibody. The humanised antibody thus closely resembles a human antibody but has the binding ability of the donor antibody.

In a further alternative, the antibody may be a “bispecific” antibody, that is an antibody having two different antigen binding domains, each domain being directed against a different epitope.

Phage display technology may be utilised to select genes which encode antibodies with binding activities towards the polypeptides of the invention either from repertoires of PCR amplified V-genes of lymphocytes from humans screened for possessing the relevant antibodies, or from naive libraries (McCafferty, J. et al., (1990), Nature 348, 552-554; Marks, J. et al., (1992) Biotechnology 10, 779-783). The affinity of these antibodies can also be improved by chain shuffling (Clackson, T. et al., (1991) Nature 352, 624-628).

Antibodies generated by the above techniques, whether polyclonal or monoclonal, have additional utility in that they may be employed as reagents in immunoassays, radioimmunoassays (RIA) or enzyme-linked immunosorbent assays (ELISA). In these applications, the antibodies can be labelled with an analytically-detectable reagent such as a radioisotope, a fluorescent molecule or an enzyme.

Preferred nucleic acid molecules of the second and third aspects of the invention are those which encode the polypeptide sequences recited in SEQ ID NO:2, and functionally equivalent polypeptides, including active fragments of the LBDS2 polypeptide, such as a fragment including the LBDS2 Nuclear Hormone Receptor Ligand Binding Domain region of the LBDS2 polypeptide sequence, or a homologue thereof.

Nucleic acid molecules encompassing these stretches of sequence form a preferred embodiment of this aspect of the invention.

These nucleic acid molecules may be used in the methods and applications described herein. The nucleic acid molecules of the invention preferably comprise at least n consecutive nucleotides from the sequences disclosed herein where, depending on the particular sequence, n is 10 or more (for example, 12, 14, 15, 18, 20, 25, 30, 35, 40 or more).

The nucleic acid molecules of the invention also include sequences that are complementary to nucleic acid molecules described above (for example, for antisense or probing purposes).

Nucleic acid molecules of the present invention may be in the form of RNA, such as mRNA, or in the form of DNA, including, for instance cDNA, synthetic DNA or genomic DNA. Such nucleic acid molecules may be obtained by cloning, by chemical synthetic techniques or by a combination thereof. The nucleic acid molecules can be prepared, for example, by chemical synthesis using techniques such as solid phase phosphoramidite chemical synthesis, from genomic or cDNA libraries or by separation from an organism. RNA molecules may generally be generated by the in vitro or in vivo transcription of DNA sequences.

The nucleic acid molecules may be double-stranded or single-stranded. Single-stranded DNA may be the coding strand, also known as the sense strand, or it may be the non-coding strand, also referred to as the anti-sense strand.

The term “nucleic acid molecule” also includes analogues of DNA and RNA, such as those containing modified backbones, and peptide nucleic acids (PNA). The term “PNA”, as used herein, refers to an antisense molecule or an anti-gene agent which comprises an oligonucleotide of at least five nucleotides in length linked to a peptide backbone of amino acid residues, which preferably ends in lysine. The terminal lysine confers solubility to the composition. PNAs may be pegylated to extend their lifespan in a cell, where they preferentially bind complementary single stranded DNA and RNA and stop transcript elongation (Nielsen, P. E. et al. (1993) Anticancer Drug Des. 8:53-63).

A nucleic acid molecule which encodes the polypeptide of SEQ ID NO:2, or an active fragment thereof, may be identical to the coding sequence of the nucleic acid molecule shown in SEQ ID NO:1. These molecules also may have a different sequence which, as a result of the degeneracy of the genetic code, encodes the polypeptide SEQ ID NO:2, or an active fragment of the LBDS2 polypeptide, such as a fragment including the LBDS2 Nuclear Hormone Receptor Ligand Binding Domain region, or a homologue thereof. The LBDS2 Nuclear Hormone Receptor Ligand Binding Domain region is considered to extend between residue 114 and residue 301 of the LBDS2 polypeptide sequence. In SEQ ID NO:1 the LBDS2 Nuclear Hormone Receptor Ligand Binding Domain region is thus encoded by a nucleic acid molecule including nucleotide 342 to 905. Nucleic acid molecules encompassing this stretch of sequence, and homologues of this sequence, form a preferred embodiment of this aspect of the invention.

Such nucleic acid molecules that encode the polypeptide of SEQ ID NO:2 may include, but are not limited to, the coding sequence for the mature polypeptide by itself; the coding sequence for the mature polypeptide and additional coding sequences, such as those encoding a leader or secretory sequence, such as a pro-, pre- or prepro-polypeptide sequence; the coding sequence of the mature polypeptide, with or without the aforementioned additional coding sequences, together with further additional, non-coding sequences, including non-coding 5′ and 3′ sequences, such as the transcribed, non-translated sequences that play a role in transcription (including termination signals), ribosome binding and mRNA stability. The nucleic acid molecules may also include additional sequences which encode additional amino acids, such as those which provide additional functionalities.

The nucleic acid molecules of the second and third aspects of the invention may also encode the fragments or the functional equivalents of the polypeptides and fragments of the first aspect of the invention.

As discussed above, a preferred fragment of the LBDS2 polypeptide is a fragment including the LBDS2 Nuclear Hormone Receptor Ligand Binding Domain region, or a homologue thereof. The Nuclear Hormone Receptor Ligand Binding Domain region is encoded by a nucleic acid molecule including nucleotide 342 to 905 of SEQ ID NO:1.

Functionally equivalent nucleic acid molecules according to the invention may be naturally-occurring variants such as a naturally-occurring allelic variant, or the molecules may be a variant that is not known to occur naturally. Such non-naturally occurring variants of the nucleic acid molecule may be made by mutagenesis techniques, including those applied to nucleic acid molecules, cells or organisms.

Among variants in this regard are variants that differ from the aforementioned nucleic acid molecules by nucleotide substitutions, deletions or insertions. The substitutions, deletions or insertions may involve one or more nucleotides. The variants may be altered in coding or non-coding regions or both. Alterations in the coding regions may produce conservative or non-conservative amino acid substitutions, deletions or insertions. The nucleic acid molecules of the invention can also be engineered, using methods generally known in the art, for a variety of reasons, including modifying the cloning, processing, and/or expression of the gene product (the polypeptide). DNA shuffling by random fragmentation and PCR reassembly of gene fragments and synthetic oligonucleotides are included as techniques which may be used to engineer the nucleotide sequences. Site-directed mutagenesis may be used to insert new restriction sites, alter glycosylation patterns, change codon preference, produce splice variants, introduce mutations and so forth.

Nucleic acid molecules which encode a polypeptide of the first aspect of the invention may be ligated to a heterologous sequence so that the combined nucleic acid molecule encodes a fusion protein. Such combined nucleic acid molecules are included within the second or third aspects of the invention. For example, to screen peptide libraries for inhibitors of the activity of the polypeptide, it may be useful to express, using such a combined nucleic acid molecule, a fusion protein that can be recognised by a commercially-available antibody. A fusion protein may also be engineered to contain a cleavage site located between the sequence of the polypeptide of the invention and the sequence of a heterologous protein so that the polypeptide may be cleaved and purified away from the heterologous protein.

The nucleic acid molecules of the invention also include antisense molecules that are partially complementary to nucleic acid molecules encoding polypeptides of the present invention and that therefore hybridize to the encoding nucleic acid molecules (hybridization). Such antisense molecules, such as oligonucleotides, can be designed to recognise, specifically bind to and prevent transcription of a target nucleic acid encoding a polypeptide of the invention, as will be known by those of ordinary skill in the art (see, for example, Cohen, J. S., Trends in Pharm. Sci., 10, 435 (1989), Okano, J. Neurochem. 56, 560 (1991); O'Connor, J. Neurochem 56, 560 (1991); Lee et al., Nucleic Acids Res 6, 3073 (1979); Cooney et al., Science 241, 456 (1988); Dervan et al., Science 251, 1360 (1991).

The term “hybridization” as used here refers to the association of two nucleic acid molecules with one another by hydrogen bonding. Typically, one molecule will be fixed to a solid support and the other will be free in solution. Then, the two molecules may be placed in contact with one another under conditions that favour hydrogen bonding. Factors that affect this bonding include: the type and volume of solvent; reaction temperature; time of hybridization; agitation; agents to block the non-specific attachment of the liquid phase molecule to the solid support (Denhardt's reagent or BLOTTO); the concentration of the molecules; use of compounds to increase the rate of association of molecules (dextran sulphate or polyethylene glycol); and the stringency of the washing conditions following hybridization (see Sambrook et al. [supra]).

The inhibition of hybridization of a completely complementary molecule to a target molecule may be examined using a hybridization assay, as known in the art (see, for example, Sambrook et al. [supra]). A substantially homologous molecule will then compete for and inhibit the binding of a completely homologous molecule to the target molecule under various conditions of stringency, as taught in Wahl, G. M. and S. L. Berger (1987; Methods Enzymol. 152:399407) and Kimmel, A. R. (1987; Methods Enzymol. 152:507-511).

“Stringency” refers to conditions in a hybridization reaction that favour the association of very similar molecules over association of molecules that differ. High stringency hybridisation conditions are defined as overnight incubation at 42° C. in a solution comprising 50% formamide, 5×SSC (150 mM NaCl, 15 mM trisodium citrate), 50 mM sodium phosphate (pH7.6), 5× Denhardts solution, 10% dextran sulphate, and 20 microgram/ml denatured, sheared salmon sperm DNA, followed by washing the filters in 0.1×SSC at approximately 65° C. Low stringency conditions involve the hybridisation reaction being carried out at 35° C. (see Sambrook et al. [supra]). Preferably, the conditions used for hybridization are those of high stringency.

Preferred embodiments of this aspect of the invention are nucleic acid molecules that are at least 80% identical over their entire length to a nucleic acid molecule encoding the LBDS2 polypeptide (SEQ ID NO:2), and nucleic acid molecules that are substantially complementary to such nucleic acid molecules. A preferred active fragment is a fragment that includes an LBDS2 Nuclear Hormone Receptor Ligand Binding Domain region of the LBDS2 polypeptide sequences, resepctively. Accordingly, preferred nucleic acid molecules include those that are at least 80% identical over their entire length to a nucleic acid molecule encoding the Nuclear Hormone Receptor Ligand Binding Domain region of the LBDS2 polypeptide sequence.

Percentage identity, as referred to herein, is as determined using BLAST version 2.1.3 using the default parameters specified by the NCBI (the National Center for Biotechnology Information; http://www.ncbi.nlm.nih.gov/).

Preferably, a nucleic acid molecule according to this aspect of the invention comprises a region that is at least 80% identical over its entire length to the nucleic acid molecule having the sequence given in SEQ ID NO:1 to a region including nucleotides 342-905 of this sequence, or a nucleic acid molecule that is complementary to any one of these regions of nucleic acid. In this regard, nucleic acid molecules at least 90%, preferably at least 95%, more preferably at least 98% or 99% identical over their entire length to the same are particularly preferred. Preferred embodiments in this respect are nucleic acid molecules that encode polypeptides which retain substantially the same biological function or activity as the LBDS2 polypeptide.

The invention also provides a process for detecting a nucleic acid molecule of the invention, comprising the steps of: (a) contacting a nucleic probe according to the invention with a biological sample under hybridizing conditions to form duplexes; and (b) detecting any such duplexes that are formed.

As discussed additionally below in connection with assays that may be utilised according to the invention, a nucleic acid molecule as described above may be used as a hybridization probe for RNA, cDNA or genomic DNA, in order to isolate full-length cDNAs and genomic clones encoding the LBDS2 polypeptide and to isolate cDNA and genomic clones of homologous or orthologous genes that have a high sequence similarity to the gene encoding this polypeptide.

In this regard, the following techniques, among others known in the art, may be utilised and are discussed below for purposes of illustration. Methods for DNA sequencing and analysis are well known and are generally available in the art and may, indeed, be used to practice many of the embodiments of the invention discussed herein. Such methods may employ such enzymes as the Klenow fragment of DNA polymerase I, Sequenase (US Biochemical Corp, Cleveland, Ohio), Taq polymerase (Perkin Elmer), thermostable T7 polymerase (Amersham, Chicago, Ill.), or combinations of polymerases and proof-reading exonucleases such as those found in the ELONGASE Amplification System marketed by Gibco/BRL (Gaithersburg, Md.). Preferably, the sequencing process may be automated using machines such as the Hamilton Micro Lab 2200 (Hamilton, Reno, Nev.), the Peltier Thermal Cycler (PTC200; M J Research, Watertown, Mass.) and the ABI Catalyst and 373 and 377 DNA Sequencers (Perkin Elmer).

One method for isolating a nucleic acid molecule encoding a polypeptide with an equivalent function to that of the LBDS2 polypeptide, particularly with an equivalent function to the LBDS2 Nuclear Hormone Receptor Ligand Binding Domain region of the LBDS2 polypeptide, is to probe a genomic or cDNA library with a natural or artificially-designed probe using standard procedures that are recognised in the art (see, for example, “Current Protocols in Molecular Biology”, Ausubel et al. (eds). Greene Publishing Association and John Wiley Interscience, New York, 1989,1992). Probes comprising at least 15, preferably at least 30, and more preferably at least 50, contiguous bases that correspond to, or are complementary to, nucleic acid sequences from the appropriate encoding gene (SEQ ID NO:1), particularly a region from nucleotides 342-905 of SEQ ID NO:1, are particularly useful probes.

Such probes may be labelled with an analytically-detectable reagent to facilitate their identification. Useful reagents include, but are not limited to, radioisotopes, fluorescent dyes and enzymes that are capable of catalysing the formation of a detectable product. Using these probes, the ordinarily skilled artisan will be capable of isolating complementary copies of genomic DNA, cDNA or RNA polynucleotides encoding proteins of interest from human, mammalian or other animal sources and screening such sources for related sequences, for example, for additional members of the family, type and/or subtype.

In many cases, isolated cDNA sequences will be incomplete, in that the region encoding the polypeptide will be cut short, normally at the 5′ end. Several methods are available to obtain full length cDNAs, or to extend short cDNAs. Such sequences may be extended utilising a partial nucleotide sequence and employing various methods known in the art to detect upstream sequences such as promoters and regulatory elements. For example, one method which may be employed is based on the method of Rapid Amplification of cDNA Ends (RACE; see, for example, Frohman et al., Proc. Natl. Acad. Sci. USA (1988) 85: 8998-9002). Recent modifications of this technique, exemplified by the Marathon™ technology (Clontech Laboratories Inc.), for example, have significantly simplified the search for longer cDNAs. A slightly different technique, termed “restriction-site” PCR, uses universal primers to retrieve unknown nucleic acid sequence adjacent a known locus (Sarkar, G. (1993) PCR Methods Applic. 2:318-322). Inverse PCR may also be used to amplify or to extend sequences using divergent primers based on a known region (Triglia, T., et al. (1988) Nucleic Acids Res. 16:8186). Another method which may be used is capture PCR which involves PCR amplification of DNA fragments adjacent a known sequence in human and yeast artificial chromosome DNA (Lagerstrom, M. et al. (1991) PCR Methods Applic. 1: 111-119). Another method which may be used to retrieve unknown sequences is that of Parker, J. D. et al. (1991); Nucleic Acids Res. 19:3055-3060). Additionally, one may use PCR, nested primers, and PromoterFinder™ libraries to walk genomic DNA (Clontech, Palo Alto, Calif.). This process avoids the need to screen libraries and is useful in finding intronlexon junctions.

When screening for full-length cDNAs, it is preferable to use libraries that have been size-selected to include larger cDNAs. Also, random-primed libraries are preferable, in that they will contain more sequences that contain the 5′ regions of genes. Use of a randomly primed library may be especially preferable for situations in which an oligo d(T) library does not yield a full-length cDNA. Genomic libraries may be useful for extension of sequence into 5′ non-transcribed regulatory regions.

In one embodiment of the invention, the nucleic acid molecules of the present invention may be used for chromosome localisation. In this technique, a nucleic acid molecule is specifically targeted to, and can hybridize with, a particular location on an individual human chromosome. The mapping of relevant sequences to chromosomes according to the present invention is an important step in the confirmatory correlation of those sequences with the gene-associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationships between genes and diseases that have been mapped to the same chromosomal region are then identified through linkage analysis (coinheritance of physically adjacent genes). This provides valuable information to investigators searching for disease genes using positional cloning or other gene discovery techniques. Once the disease or syndrome has been crudely localised by genetic linkage to a particular genomic region, any sequences mapping to that area may represent associated or regulatory genes for further investigation. The nucleic acid molecule may also be used to detect differences in the chromosomal location due to translocation, inversion, etc. among normal, carrier, or affected individuals.

The nucleic acid molecules of the present invention are also valuable for tissue localisation. Such techniques allow the determination of expression patterns of the polypeptide in tissues by detection of the mRNAs that encode them. These techniques include in situ hybridization techniques and nucleotide amplification techniques, such as PCR. Results from these studies provide an indication of the normal functions of the polypeptide in the organism. In addition, comparative studies of the normal expression pattern of mRNAs with that of mRNAs encoded by a mutant gene provide valuable insights into the role of mutant polypeptides in disease. Such inappropriate expression may be of a temporal, spatial or quantitative nature.

The vectors of the present invention comprise nucleic acid molecules of the invention and may be cloning or expression vectors. The host cells of the invention, which may be transformed, transfested or transduced with the vectors of the invention may be prokaryotic or eukaryotic.

The polypeptides of the invention may be prepared in recombinant form by expression of their encoding nucleic acid molecules in vectors contained within a host cell. Such expression methods are well known to those of skill in the art and many are described in detail by Sambrook et al. (supra) and Fernandez & Hoeffler (1998, eds. “Gene expression systems. Using nature for the art of expression”. Academic Press, San Diego, London, Boston, New York, Sydney, Tokyo, Toronto).

Generally, any system or vector that is suitable to maintain, propagate or express nucleic acid molecules to produce a polypeptide in the required host may be used. The appropriate nucleotide sequence may be inserted into an expression system by any of a variety of well-known and routine techniques, such as, for example, those described in Sambrook et al., (supra). Generally, the encoding gene can be placed under the control of a control element such as a promoter, ribosome binding site (for bacterial expression) and, optionally, an operator, so that the DNA sequence encoding the desired polypeptide is transcribed into RNA in the transformed host cell.

Examples of suitable expression systems include, for example, chromosomal, episomal and virus-derived systems, including, for example, vectors derived from: bacterial plasmids, bacteriophage, transposons, yeast episomes, insertion elements, yeast chromosomal elements, viruses such as baculoviruses, papova viruses such as SV40, vaccinia viruses, adenoviruses, fowl pox viruses, pseudorabies viruses and retroviruses, or combinations thereof, such as those derived from plasmid and bacteriophage genetic elements, including cosmids and phagemids. Human artificial chromosomes (HACs) may also be employed to deliver larger fragments of DNA than can be contained and expressed in a plasmid.

Particularly suitable expression systems include microorganisms such as bacteria transformed with recombinant bacteriophage, plasmid or cosmid DNA expression vectors; yeast transformed with yeast expression vectors; insect cell systems infected with virus expression vectors (for example, baculovirus); plant cell systems transformed with virus expression vectors (for example, cauliflower mosaic virus, CaMV; tobacco mosaic virus, TMV) or with bacterial expression vectors (for example, Ti or pBR322 plasmids); or animal cell systems. Cell-free translation systems can also be employed to produce the polypeptides of the invention.

Introduction of nucleic acid molecules encoding a polypeptide of the present invention into host cells can be effected by methods described in many standard laboratory manuals, such as Davis et al., Basic Methods in Molecular Biology (1986) and Sambrook et al., (supra). Particularly suitable methods include calcium phosphate transfection, DEAE-dextran mediated transfection, transvection, microinjection, cationic lipid-mediated transfection, electroporation, transduction, scrape loading, ballistic introduction or infection (see Sambrook et al., 1989 [supra]; Ausubel et al., 1991 [supra]; Spector, Goldman & Leinwald, 1998). In eukaryotic cells, expression systems may either be transient (for example, episomal) or permanent (chromosomal integration) according to the needs of the system.

The encoding nucleic acid molecule may or may not include a sequence encoding a control sequence, such as a signal peptide or leader sequence, as desired, for example, for secretion of the translated polypeptide into the lumen of the endoplasmic reticulum, into the periplasmic space or into the extracellular environment. These signals may be endogenous to the polypeptide or they may be heterologous signals. Leader sequences can be removed by the bacterial host in post-translational processing.

In addition to control sequences, it may be desirable to add regulatory sequences that allow for regulation of the expression of the polypeptide relative to the growth of the host cell. Examples of regulatory sequences are those which cause the expression of a gene to be increased or decreased in response to a chemical or physical stimulus, including the presence of a regulatory compound or to various temperature or metabolic conditions. Regulatory sequences are those non-translated regions of the vector, such as enhancers, promoters and 5′ and 3′ untranslated regions. These interact with host cellular proteins to carry out transcription and translation. Such regulatory sequences may vary in their strength and specificity. Depending on the vector system and host utilised, any number of suitable transcription and translation elements, including constitutive and inducible promoters, may be used. For example, when cloning in bacterial systems, inducible promoters such as the hybrid lacZ promoter of the Bluescript phagemid (Stratagene, LaJolla, Calif.) or pSportl™ plasmid (Gibco BRL) and the like may be used. The baculovirus polyhedrin promoter may be used in insect cells. Promoters or enhancers derived from the genomes of plant cells (for example, heat shock, RUBISCO and storage protein genes) or from plant viruses (for example, viral promoters or leader sequences) may be cloned into the vector. In mammalian cell systems, promoters from mammalian genes or from mammalian viruses are preferable. If it is necessary to generate a cell line that contains multiple copies of the sequence, vectors based on SV40 or EBV may be used with an appropriate selectable marker.

An expression vector is constructed so that the particular nucleic acid coding sequence is located in the vector with the appropriate regulatory sequences, the positioning and orientation of the coding sequence with respect to the regulatory sequences being such that the coding sequence is transcribed under the “control” of the regulatory sequences, i.e., RNA polymerase which binds to the DNA molecule at the control sequences transcribes the coding sequence. In some cases it may be necessary to modify the sequence so that it may be attached to the control sequences with the appropriate orientation; i.e., to maintain the reading frame.

The control sequences and other regulatory sequences may be ligated to the nucleic acid coding sequence prior to insertion into a vector. Alternatively, the coding sequence can be cloned directly into an expression vector that already contains the control sequences and an appropriate restriction site.

For long-term, high-yield production of a recombinant polypeptide, stable expression is preferred. For example, cell lines which stably express the polypeptide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type.

Mammalian cell lines available as hosts for expression are known in the art and include many immortalised cell lines available from the American Type Culture Collection (ATCC) including, but not limited to, Chinese hamster ovary (CHO), HeLa, baby hamster kidney (BHK), monkey kidney (COS), C127, 3T3, BHK, HEK 293, Bowes melanoma and human hepatocellular carcinoma (for example Hep G2) cells and a number of other cell lines.

In the baculovirus system, the materials for baculovirus/insect cell expression systems are commercially available in kit form from, inter alia, Invitrogen, San Diego Calif. (the “MaxBac” kit). These techniques are generally known to those skilled in the art and are described fully in Summers and Smith, Texas Agricultural Experiment Station Bulletin No. 1555 (1987). Particularly suitable host cells for use in this system include insect cells such as Drosophila S2 and Spodoptera Sf9 cells.

There are many plant cell culture and whole plant genetic expression systems known in the art. Examples of suitable plant cellular genetic expression systems include those described in U.S. Pat. Nos. 5,693,506; 5,659,122; and 5,608,143. Additional examples of genetic expression in plant cell culture has been described by Zenk, (1991) Phytochemistry 30, 3861-3863.

In particular, all plants from which protoplasts can be isolated and cultured to give whole regenerated plants can be utilised, so that whole plants are recovered which contain the transferred gene. Practically all plants can be regenerated from cultured cells or tissues, including but not limited to all major species of sugar cane, sugar beet, cotton, fruit and other trees, legumes and vegetables.

Examples of particularly preferred bacterial host cells include streptococci, staphylococci, E. coli, Streptomyces and Bacillus subtilis cells.

Examples of particularly suitable host cells for fungal expression include yeast cells (for example, S. cerevisiae) and Aspergillus cells.

Any number of selection systems are known in the art that may be used to recover transformed cell lines. Examples include the herpes simplex virus thymidine kinase (Wigler, M. et al. (1977) Cell 11:223-32) and adenine phosphoribosyltransferase (Lowy, I. et al. (1980) Cell 22:817-23) genes that can be employed in tk- or aprt± cells, respectively.

Also, antimetabolite, antibiotic or herbicide resistance can be used as the basis for selection; for example, dihydrofolate reductase (DHFR) that confers resistance to methotrexate (Wigler, M. et al. (1980) Proc. Nad. Acad. Sci. 77:3567-70); npt, which confers resistance to the aminoglycosides neomycin and G-418 (Colbere-Garapin, F. et al. (1981) J. Mol. Biol. 150:1-14) and als or pat, which confer resistance to chlorsulfuron and phosphinotricin acetyltransferase, respectively. Additional selectable genes have been described, examples of which will be clear to those of skill in the art.

Although the presence or absence of marker gene expression suggests that the gene of interest is also present, its presence and expression may need to be confirmed. For example, if the relevant sequence is inserted within a marker gene sequence, transformed cells containing the appropriate sequences can be identified by the absence of marker gene function. Alternatively, a marker gene can be placed in tandem with a sequence encoding a polypeptide of the invention under the control of a single prompter. Expression of the marker gene in response to induction or selection usually indicates expression of the tandem gene as well.

Alternatively, host cells that contain a nucleic acid sequence encoding a polypeptide of the invention and which express said polypeptide may be identified by a variety of procedures known to those of skill in the art. These procedures include, but are not limited to, DNA-DNA or DNA-RNA hybridizations and protein bioassays, for example, fluorescence activated cell sorting (FACS) or immunoassay techniques (such as the enzyme-linked immunosorbent assay [ELISA] and radioimmunoassay [RIA]), that include membrane, solution, or chip based technologies for the detection and/or quantification of nucleic acid or protein (see Hampton, R. et al. (1990) Serological Methods, a Laboratory Manual, APS Press, St Paul, Minn.) and Maddox, D. E. et al. (1983) J. Exp. Med, 158, 1211-1216).

A wide variety of labels and conjugation techniques are known by those skilled in the art and may be used in various nucleic acid and amino acid assays. Means for producing labelled hybridization or PCR probes for detecting sequences related to nucleic acid molecules encoding polypeptides of the present invention include oligolabelling, nick translation, end-labelling or PCR amplification using a labelled polynucleotide. Alternatively, the sequences encoding the polypeptide of the invention may be cloned into a vector for the production of an mRNA probe. Such vectors are known in the art, are commercially available, and may be used to synthesise RNA probes in vitro by addition of an appropriate RNA polymerase such as T7, T3 or SP6 and labelled nucleotides. These procedures may be conducted using a variety of commercially available kits (Pharmacia & Upjohn, (Kalamazoo, Mich.); Promega (Madison Wis.; and U.S. Biochemical Corp., Cleveland, Ohio)).

Suitable reporter molecules or labels, which may be used for ease of detection, include radionuclides, enzymes and fluorescent, chemilurninescent or chromogenic agents as well as substrates, cofactors, inhibitors, magnetic particles, and the like.

Nucleic acid molecules according to the present invention may also be used to create transgenic animals, particularly rodent animals. Such transgenic animals form a further aspect of the present invention. This may be done locally by modification of somatic cells, or by germ line therapy to incorporate heritable modifications. Such transgenic animals may be particularly useful in the generation of animal models for drug molecules effective as modulators of the polypeptides of the present invention.

The polypeptide can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography is particularly useful for purification. Well known techniques for refolding proteins may be employed to regenerate an active conformation when the polypeptide is denatured during isolation and or purification.

Specialised vector constructions may also be used to facilitate purification of proteins, as desired, by joining sequences encoding the polypeptides of the invention to a nucleotide sequence encoding a polypeptide domain that will facilitate purification of soluble proteins. Examples of such purification-facilitating domains include metal chelating peptides such as histidine-tryptophan modules that allow purification on immobilised metals, protein A domains that allow purification on immobilised immunoglobulin, and the domain utilised in the FLAGS extension/affinity purification system (Immunex Corp., Seattle, Wash.). The inclusion of cleavable linker sequences such as those specific for Factor XA or enterokinase (Invitrogen, San Diego, Calif.) between the purification domain and the polypeptide of the invention may be used to facilitate purification. One such expression vector provides for expression of a fusion protein containing the polypeptide of the invention fused to several histidine residues preceding a thioredoxin or an enterokinase cleavage site. The histidine residues facilitate purification by IMAC (immobilised metal ion affinity chromatography as described in Porath, J. et al. (1992) Prot. Exp. Purif. 3: 263-281) while the thioredoxin or enterokinase cleavage site provides a means for purifying the polypeptide from the fusion protein. A discussion of vectors which contain fusion proteins is provided in Kroll, D. J. et al. (DNA Cell Biol. 199312:441-453).

If the polypeptide is to be expressed for use in screening assays, generally it is preferred that it be produced at the surface of the host cell in which it is expressed. In this event, the host cells may be harvested prior to use in the screening assay, for example using techniques such as fluorescence activated cell sorting (FACS) or immunoaffinity techniques. If the polypeptide is secreted into the medium, the medium can be recovered in order to recover and purify the expressed polypeptide. If polypeptide is produced intracellularly, the cells must first be lysed before the polypeptide is recovered.

The polypeptide of the invention can be used to screen libraries of compounds in any of a variety of drug screening techniques. Such compounds may activate (agonise) or inhibit (antagonise) the level of expression of the gene or the activity of the polypeptide of the invention and form a further aspect of the present invention. Preferred compounds are effective to alter the expression of a natural gene which encodes a polypeptide of the first aspect of the invention or to regulate the activity of a polypeptide of the first aspect of the invention.

Agonist or antagonist compounds may be isolated from, for example, cells, cell-free preparations, chemical libraries or natural product mixtures. These agonists or antagonists may be natural or modified substrates, ligands, enzymes, receptors or structural or functional mimetics. For a suitable review of such screening techniques, see Coligan et al., Current Protocols in Immunology 1(2):Chapter 5 (1991).

Compounds that are most likely to be good antagonists are molecules that bind to the polypeptide of the invention without inducing the biological effects of the polypeptide upon binding to it. Potential antagonists include small organic molecules, peptides, polypeptides and antibodies that bind to the polypeptide of the invention and thereby inhibit or extinguish its activity. In this fashion, binding of the polypeptide to normal cellular binding molecules may be inhibited, such that the normal biological activity of the polypeptide is prevented.

The polypeptide of the invention that is employed in such a screening technique may be free in solution, affixed to a solid support, borne on a cell surface or located intracellularly. In general, such screening procedures may involve using appropriate cells or cell membranes that express the polypeptide that are contacted with a test compound to observe binding, or stimulation or inhibition of a functional response. The functional response of the cells contacted with the test compound is then compared with control cells that were not contacted with the test compound. Such an assay may assess whether the test compound results in a signal generated by activation of the polypeptide, using an appropriate detection system. Inhibitors of activation are generally assayed in the presence of a known agonist and the effect on activation by the agonist in the presence of the test compound is observed.

Alternatively, simple binding assays may be used, in which the adherence of a test compound to a surface bearing the polypeptide is detected by means of a label directly or indirectly associated with the test compound or in an assay involving competition with a labelled competitor. In another embodiment, competitive drug screening assays may be used, in which neutralising antibodies that are capable of binding the polypeptide specifically compete with a test compound for binding. In this manner, the antibodies can be used to detect the presence of any test compound that possesses specific binding affinity for the polypeptide.

Assays may also be designed to detect the effect of added test compounds on the production of mRNA encoding the polypeptide in cells. For example, an ELISA may be constructed that measures secreted or cell-associated levels of polypeptide using monoclonal or polyclonal antibodies by standard methods known in the art, and this can be used to search for compounds that may inhibit or enhance the production of the polypeptide from suitably manipulated cells or tissues. The formation of binding complexes between the polypeptide and the compound being tested may then be measured.

Another technique for drug screening which may be used provides for high throughput screening of compounds having suitable binding affinity to the polypeptide of interest (see International patent application WO84/03564). In this method, large numbers of different small test compounds are synthesised on a solid substrate, which may then be reacted with the polypeptide of the invention and washed. One way of immobilising the polypeptide is to use non-neutralising antibodies. Bound polypeptide may then be detected using methods that are well known in the art. Purified polypeptide can also be coated directly onto plates for use in the aforementioned drug screening techniques.

The polypeptide of the invention may be used to identify membrane-bound or soluble receptors, through standard receptor binding techniques that are known in the art, such as ligand binding and crosslinking assays in which the polypeptide is labelled with a radioactive isotope, is chemically modified, or is fused to a peptide sequence that facilitates its detection or purification, and incubated with a source of the putative receptor (for example, a composition of cells, cell membranes, cell supernatants, tissue extracts, or bodily fluids). The efficacy of binding may be measured using biophysical techniques such as surface plasmon resonance and spectroscopy. Binding assays may be used for the purification and cloning of the receptor, but may also identify agonists and antagonists of the polypeptide, that compete with the binding of the polypeptide to its receptor. Standard methods for conducting screening assays are well understood in the art.

The invention also includes a screening kit useful in the methods for identifying agonists, antagonists, ligands, receptors, substrates, enzymes, that are described above.

The invention includes the agonists, antagonists, ligands, receptors, substrates and enzymes, and other compounds which modulate the activity or antigenicity of the polypeptide of the invention discovered by the methods that are described above.

The invention also provides pharmaceutical compositions comprising a polypeptide, nucleic acid, ligand or compound of the invention in combination with a suitable pharmaceutical carrier. These compositions may be suitable as therapeutic or diagnostic reagents, as vaccines, or as other immunogenic compositions, as outlined in detail below.

According to the terminology used herein, a composition containing a polypeptide, nucleic acid, ligand or compound [X] is “substantially free of” impurities [herein, Y] when at least 85% by weight of the total X+Y in the composition is X. Preferably, X comprises at least about 90% by weight of the total of X+Y in the composition, more preferably at least about 95%, 98% or even 99% by weight.

The pharmaceutical compositions should preferably comprise a therapeutically effective amount of the polypeptide, nucleic acid molecule, ligand, or compound of the invention. The term “therapeutically effective amount” as used herein refers to an amount of a therapeutic agent needed to treat, ameliorate, or prevent a targetted disease or condition, or to exhibit a detectable therapeutic or preventative effect. For any compound, the therapeutically effective dose can be estimated initially either in cell culture assays, for example, of neoplastic cells, or in animal models, usually mice, rabbits, dogs, or pigs. The animal model may also be used to determine the appropriate concentration range and route of administration. Such information can then be used to determine useful doses and routes for administration in humans.

The precise effective amount for a human subject will depend upon the severity of the disease state, general health of the subject, age, weight, and gender of the subject, diet, time and frequency of administration, drug combination(s); reaction sensitivities, and tolerance/response to therapy. This amount can be determined by routine experimentation and is within the judgement of the clinician. Generally, an effective dose will be from 0.01 mg/kg to 50 mg/kg, preferably 0.05 mg/kg to 10 mg/kg. Compositions may be administered individually to a patient or may be administered in combination with other agents, drugs or hormones.

A pharmaceutical composition may also contain a pharmaceutically acceptable carrier, for administration of a therapeutic agent. Such carriers include antibodies and other polypeptides, genes and other therapeutic agents such as liposomes, provided that the carrier does not itself induce the production of antibodies harmful to the individual receiving the composition, and which may be administered without undue toxicity. Suitable carriers may be large, slowly metabolised macromolecules such as proteins, polysaccharides, polylactic acids, polyglycolic acids, polymeric amino acids, amino acid copolymers and inactive virus particles.

Pharmaceutically acceptable salts can be used therein, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable carriers is available in Remington's Pharmaceutical Sciences (Mack Pub. Co., N.J. 1991).

Pharmaceutically acceptable carriers in therapeutic compositions may additionally contain liquids such as water, saline, glycerol and ethanol. Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient.

Once formulated, the compositions of the invention can be administered directly to the subject. The subjects to be treated can be animals; in particular, human subjects can be treated.

The pharmaceutical compositions utilised in this invention may be administered by any number of routes including, but not limited to, oral, intravenous, intramuscular, intra-arterial, intramedullary, intrathecal, intraventricular, transdermal or transcutaneous applications (for example, see WO98/20734), subcutaneous, intraperitoneal, intranasal, enteral, topical, sublingual, intravaginal or rectal means. Gene guns or hyposprays may also be used to administer the pharmaceutical compositions of the invention. Typically, the therapeutic compositions may be prepared as injectables, either as liquid solutions or suspensions; solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule.

If the activity of the polypeptide of the invention is in excess in a particular disease state, several approaches are available. One approach comprises administering to a subject an inhibitor compound (antagonist) as described above, along with a pharmaceutically acceptable carrier in an amount effective to inhibit the function of the polypeptide, such as by blocking the binding of ligands, substrates, enzymes, receptors, or by inhibiting a second signal, and thereby alleviating the abnormal condition. Preferably, such antagonists are antibodies. Most preferably, such antibodies are chimeric and/or humanised to minimise their immunogenicity, as described previously.

In another approach, soluble forms of the polypeptide that retain binding affinity for the ligand, substrate, enzyme, receptor, in question, may be administered. Typically, the polypeptide may be administered in the form of fragments that retain the relevant portions.

In an alternative approach, expression of the gene encoding the polypeptide can be inhibited using expression blocking techniques, such as the use of antisense nucleic acid molecules (as described above), either internally generated or separately administered. Modifications of gene expression can be obtained by designing complementary sequences or antisense molecules (DNA, RNA, or PNA) to the control, 5′ or regulatory regions (signal sequence, promoters, enhancers and introns) of the gene encoding the polypeptide. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Carr, Molecular and Irmunologic Approaches, Futura Publishing Co., Mt. Kisco, N.Y.). The complementary sequence or antisense molecule may also be designed to block translation of mRNA by preventing the transcript from binding to ribosomes. Such oligonucleotides may be administered or may be generated in situ from expression in vivo.

In addition, expression of the polypeptide of the invention may be prevented by using ribozymes specific to its encoding mRNA sequence. Ribozymes are catalytically active RNAs that can be natural or synthetic (see for example Usman, N, et al., Curr. Opin. Struct. Biol (1996) 6(4), 527-33). Synthetic ribozymes can be designed to specifically cleave mRNAs at selected positions thereby preventing translation of the mRNAs into functional polypeptide. Ribozymes may be synthesised with a natural ribose phosphate backbone and natural bases, as normally found in RNA molecules. Alternatively the ribozymes may be synthesised with non-natural backbones, for example, 2′-O-methyl RNA, to provide protection from ribonuclease degradation and may contain modified bases.

RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine and butosine, as well as acetyl-, methyl-, thio- and similarly modified forms of adenine, cytidine, guanine, thymine and uridine which are not as easily recognised by endogenous endonucleases.

For treating abnormal conditions related to an under-expression of the polypeptide of the invention and its activity, several approaches are also available. One approach comprises administering to a subject a therapeutically effective amount of a compound that activates the polypeptide, i.e., an agonist as described above, to alleviate the abnormal condition. Alternatively, a therapeutic amount of the polypeptide in combination with a suitable pharmaceutical carrier may be administered to restore the relevant physiological balance of polypeptide.

Gene therapy may be employed to effect the endogenous production of the polypeptide by the relevant cells in the subject. Gene therapy is used to treat permanently the inappropriate production of the polypeptide by replacing a defective gene with a corrected therapeutic gene.

Gene therapy of the present invention can occur in vivo or ex vivo. Ex vivo gene therapy requires the isolation and purification of patient cells, the introduction of a therapeutic gene and introduction of the genetically altered cells back into the patient. In contrast, in vivo gene therapy does not require isolation and purification of a patient's cells.

The therapeutic gene is typically “packaged” for administration to a patient. Gene delivery vehicles may be non-viral, such as liposomes, or replication-deficient viruses, such as adenovirus as described by Berkner, K. L., in Curr. Top. Microbiol. Immunol., 158, 39-66 (1992) or adeno-associated virus (AAV) vectors as described by Muzyczka, N., in Curr. Top. Microbiol. Immunol., 158, 97-129 (1992) and U.S. Pat. No. 5,252,479. For example, a nucleic acid molecule encoding a polypeptide of the invention may be engineered for expression in a replication-defective retroviral vector. This expression construct may then be isolated and introduced into a packaging cell transduced with a retroviral plasmid vector containing RNA encoding the polypeptide, such that the packaging cell now produces infectious viral particles containing the gene of interest. These producer cells may be administered to a subject for engineering cells in vivo and expression of the polypeptide in vivo (see Chapter 20, Gene Therapy and other Molecular Genetic-based Therapeutic Approaches, (and references cited therein) in Human Molecular Genetics (1996), T Strachan and A P Read, BIOS Scientific Publishers Ltd).

Another approach is the administration of “naked DNA” in which the therapeutic gene is directly injected into the bloodstream or muscle tissue.

In situations in which the polypeptides or nucleic acid molecules of the invention are disease-causing agents, the invention provides that they can be used in vaccines to raise antibodies against the disease causing agent.

Vaccines according to the invention may either be prophylactic (ie. to prevent infection) or therapeutic (ie. to treat disease after infection). Such vaccines comprise immunising antigen(s), immunogen(s), polypeptide(s), protein(s) or nucleic acid, usually in combination with pharmaceutically-acceptable carriers as described above, which include any carrier that does not itself induce the production of antibodies harmful to the individual receiving the composition. Additionally, these carriers may function as immunostimulating agents (“adjuvants”). Furthermore, the antigen or immunogen may be conjugated to a bacterial toxoid, such as a toxoid from diphtheria, tetanus, cholera, H. pylon, and other pathogens.

Since polypeptides may be broken down in the stomach, vaccines comprising polypeptides are preferably administered parenterally (for instance, subcutaneous, intramuscular, intravenous, or intradermal injection). Formulations suitable for parenteral administration include aqueous and non-aqueous sterile injection solutions which may contain anti-oxidants, buffers, bacteriostats and solutes which render the formulation isotonic with the blood of the recipient, and aqueous and non-aqueous sterile suspensions which may include suspending agents or thickening agents.

The vaccine formulations of the invention may be presented in unit-dose or multi-dose containers. For example, sealed ampoules and vials and may be stored in a freeze-dried condition requiring only the addition of the sterile liquid carrier immediately prior to use. The dosage will depend on the specific activity of the vaccine and can be readily determined by routine experimentation.

This invention also relates to the use of nucleic acid molecules according to the present invention as diagnostic reagents. Detection of a mutated form of the gene characterised by the nucleic acid molecules of the invention which is associated with a dysfunction win provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques.

Nucleic acid molecules for diagnosis may be obtained from a subject's cells, such as from blood, urine, saliva, tissue biopsy or autopsy material. The genomic DNA may be used directly for detection or may be amplified enzymatically by using PCR, ligase chain reaction (LCR), strand displacement amplification (SDA), or other amplification techniques (see Saiki et al., Nature, 324, 163-166 (1986); Bej, et al., Crit Rev. Biochem. Molec. Biol., 26, 301-334 (1991); Birkenmeyer et al., J. Virol. Meth., 35, 117-126 (1991); Van Brunt, J., Bio/Technology, 8, 291-294 (1990)) prior to analysis.

In one embodiment, this aspect of the invention provides a method of diagnosing a disease in a patient, comprising assessing the level of expression of a natural gene encoding a polypeptide according to the invention and comparing said level of expression to a control level, wherein a level that is different to said control level is indicative of disease. The method may comprise the steps of:

a) contacting a sample of tissue from the patient with a nucleic acid probe under stringent conditions that allow the formation of a hybrid complex between a nucleic acid molecule of the invention and the probe;

b) contacting a control sample with said probe under the same conditions used in step a);

c) and detecting the presence of hybrid complexes in said samples;

wherein detection of levels of the hybrid complex in the patient sample that differ from levels of the hybrid complex in the control sample is indicative of disease.

A further aspect of the invention comprises a diagnostic method comprising the steps of:

a) obtaining a tissue sample from a patient being tested for disease;

b) isolating a nucleic acid molecule according to the invention from said tissue sample; and,

c) diagnosing the patient for disease by detecting the presence of a mutation in the nucleic acid molecule which is associated with disease.

To aid the detection of nucleic acid molecules in the above-described methods, an amplification step, for example using PCR, may be included.

Deletions and insertions can be detected by a change in the size of the amplified product in comparison to the normal genotype. Point mutations can be identified by hybridizing amplified DNA to labelled RNA of the invention or alternatively, labelled antisense DNA sequences of the invention. Perfectly-matched sequences can be distinguished from mismatched duplexes by RNase digestion or by assessing differences in melting temperatures. The presence or absence of the mutation in the patient may be detected by contacting DNA with a nucleic acid probe that hybridises to the DNA under stringent conditions to form a hybrid double-stranded molecule, the hybrid double-stranded molecule having an unhybridised portion of the nucleic acid probe strand at any portion corresponding to a mutation associated with disease; and detecting the presence or absence of an unhybridised portion of the probe strand as an indication of the presence or absence of a disease-associated mutation in the corresponding portion of the DNA strand.

Such diagnostics are particularly useful for prenatal and even neonatal testing.

Point mutations and other sequence differences between the reference gene and “mutant” genes can be identified by other well-known techniques, such as direct DNA sequencing or single-strand conformational polymorphism, (see Orita et al., Genomics, 5, 874-879 (1989)). For example, a sequencing primer may be used with double-stranded PCR product or a single-stranded template molecule generated by a modified PCR. The sequence determination is performed by conventional procedures with radiolabelled nucleotides or by automatic sequencing procedures with fluorescent-tags. Cloned DNA segments may also be used as probes to detect specific DNA segments. The sensitivity of this method is greatly enhanced when combined with PCR. Further, point mutations and other sequence variations, such as polymorphisms, can be detected as described above, for example, through the use of allele-specific oligonucleotides for PCR amplification of sequences that differ by single nucleotides.

DNA sequence differences may also be detected by alterations in the electrophoretic mobility of DNA fragments in gels, with or without denaturing agents, or by direct DNA sequencing (for example, Myers et al., Science (1985) 230:1242). Sequence changes at specific locations may also be revealed by nuclease protection. assays, such as RNase and S1 protection or the chemical cleavage method (see Cotton et al., Proc. Natl. Acad. Sci. USA (1985) 85: 4397-4401).

In addition to conventional gel electrophoresis and DNA sequencing, mutations such as microdeletions, aneuploidies, translocations, inversions, can also be detected by in situ analysis (see, for example, Keller et al., DNA Probes, 2nd Ed., Stockton Press, New York, N.Y., USA (1993)), that is, DNA or RNA sequences in cells can be analysed for mutations without need for their isolation and/or immobilisation onto a membrane. Fluorescence in situ hybridization (FISH) is presently the most commonly applied method and numerous reviews of FISH have appeared (see, for example, Trachuck et al., Science, 250: 559-562 (1990), and Trask et al., Trends, Genet. 7:149-154 (1991)).

In another embodiment of the invention, an array of oligonucleotide probes comprising a nucleic acid molecule according to the invention can be constructed to conduct efficient screening of genetic variants, mutations and polymorphisms. Array technology methods are well known and have general applicability and can be used to address a variety of questions in molecular genetics including gene expression, genetic linkage, and genetic variability (see for example: M. Chee et al., Science (1996) 274: 610-613).

In one embodiment, the array is prepared and used according to the methods described in PCT application WO95/11995 (Chee et al); Lockhart, D. J. et al. (1996) Nat. Biotech. 14: 1675-1680); and Schena, M. et al. (1996) Proc. Natl. Acad. Sci. 93: 10614-10619). Oligonucleotide pairs may range from two to over one million. The oligomers are synthesized at designated areas on a substrate using a light-directed chemical process. The substrate may be paper, nylon or other type of membrane, filter, chip, glass slide or any other suitable solid support. In another aspect, an oligonucleotide may be synthesized on the surface of the substrate by using a chemical coupling procedure and an ink jet application apparatus, as described in PCT application WO95/251116 (13aldeschweiler et al.). In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536 or 6144 oligonucleotides, or any other number between two and over one million which lends itself to the efficient use of commercially-available instrumentation.

In addition to the methods discussed above, diseases may be diagnosed by methods comprising determining from a sample derived from a subject, an abnormally decreased or increased level of polypeptide or mRNA. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods.

Assay techniques that can be used to determine levels of a polypeptide of the present invention in a sample derived from a host are well-known to those of skill in the art and are discussed in some detail above (including radioimmunoassays, competitive-binding assays, Western Blot analysis and ELISA assays). This aspect of the invention provides a diagnostic method which comprises the steps of: (a) contacting a ligand as described above with a biological sample under conditions suitable for the formation of a ligand-polypeptide complex; and (b) detecting said complex.

Protocols such as ELISA, RIA, and FACS for measuring polypeptide levels may additionally provide a basis for diagnosing altered or abnormal levels of polypeptide expression. Normal or standard values for polypeptide expression are established by combining body fluids or cell extracts taken from normal mammalian subjects, preferably humans, with antibody to the polypeptide under conditions suitable for complex formation The amount of standard complex formation may be quantified by various methods, such as by photometric means.

Antibodies which specifically bind to a polypeptide of the invention may be used for the diagnosis of conditions or diseases characterised by expression of the polypeptide, or in assays to monitor patients being treated with the polypeptides, nucleic acid molecules, ligands and other compounds of the invention. Antibodies useful for diagnostic purposes may be prepared in the same manner as those described above for therapeutics. Diagnostic assays for the polypeptide include methods that utilise the antibody and a label to detect the polypeptide in human body fluids or extracts of cells or tissues. The antibodies may be used with or without modification, and may be labelled by joining them, either covalently or non-covalently, with a reporter molecule. A wide variety of reporter molecules known in the art may be used, several of which are described above.

Quantities of polypeptide expressed in subject, control and disease samples from biopsied tissues are compared with the standard values. Deviation between standard and subject values establishes the parameters for diagnosing disease. Diagnostic assays may be used to distinguish between absence, presence, and excess expression of polypeptide and to monitor regulation of polypeptide levels during therapeutic intervention. Such assays may also be used to evaluate the efficacy of a particular therapeutic treatment regimen in animal studies, in clinical trials or in monitoring the treatment of an individual patient.

A diagnostic kit of the present invention may comprise:

(a) a nucleic acid molecule of the present invention;

(b) a polypeptide of the present invention; or

(c) a ligand of the present invention.

In one aspect of the invention, a diagnostic kit may comprise a first container containing a nucleic acid probe that hybridises under stringent conditions with a nucleic acid molecule according to the invention; a second container containing primers useful for amplifying the nucleic acid molecule; and instructions for using the probe and primers for facilitating the diagnosis of disease. The kit may further comprise a third container holding an agent for digesting unhybridised RNA.

In an alternative aspect of the invention, a diagnostic kit may comprise an array of nucleic acid molecules, at least one of which may be a nucleic acid molecule according to the invention.

To detect polypeptide according to the invention, a diagnostic kit may comprise one or more antibodies that bind to a polypeptide according to the invention; and a reagent useful for the detection of a binding reaction between the antibody and the polypeptide.

Such kits will be of use in diagnosing a disease or susceptibility to disease, particularly cell proliferative disorders, including neoplasm, cancer and myeloproliferative disorders and angiogenesis, metabolic disorders, including obesity, osteoporosis, non-insulin dependent diabetes, lipid metabolism disorder, hyperthyroidism, hyperparathyroidism, hypercalcemia, hypercholestrolemia, hyperlipidemia, cardiovascular disorders including, hypertension, athersclerosis, hyperinsulinemia, heart arrhythmia, renal disorders, including glomerulonephritis, renovascular hypertension, skin disorders including dermatological disease, psoriasis, acne, aging, eczema, wound healing, inflammation, including inflammatory bowel disease, respiratory tract inflammation, emphysema, asthma, immune disorder, autoimmune disease, multiple sclerosis, nervous system disorders, including anxiety, depression, neurodegenerative disease, Alzheimer's disease Parkinson's disease, brain injury, stroke and pain, infection, including virus infection, and other conditions in which nuclear hormones are implicated.

Various aspects and embodiments of the present invention will now be described in more detail by way of example, with particular reference to the LBDS2 polypeptide.

It will be appreciated that modification of detail may be made without departing from the scope of the invention.

### BRIEF DESCRIPTION OF THE FIGURES

FIG. 1: This is the front end of the Biopendium Target Mining Interface. A search of the database is initiated using the PDB code “1QKN:A”.

FIG. 2A: A selection is shown of the Inpharmatica Genome Threader results for the search using 1QKN:A. The arrow indicates the Homo Sapiens Estrogen Receptor, which has a typical Nuclear Hormone Receptor Ligand Binding Domain.

FIG. 2B: A selection is shown of the Inpharmatica Genome Threader results for the search using 1QKN:A. The arrow indicates BAA74848.1 (LBDS2).

FIG. 2C: Full list of forward PSI-BLAST results for the search using 1QKN:A. BAA74848.1 (LBDS2) is not identified.

FIG. 3: The Redundant Sequence Display results page for BAA74848.1 (LBDS2).

FIG. 4: InterPro PFAM search results for BAA74848.1 (LBDS2).

FIG. 5: NCBI Protein Report for BAA74848.1 (LBDS2).

FIG. 6A: This is the front end of the Biopendium database. A search of the database is initiated using BAA74848.1 (LBDS2), as the query sequence.

FIG. 6B: A selection of the Inpharmatica Genome Threader results of search using BAA74848.1 (LBDS2), as the query sequence. The arrow points to 1QKN:A.

FIG. 6C: The reverse-maximised PSI-BLAST results obtained using BAA74848.1 (IBDS2), as the query sequence.

FIG. 7: AlEye sequence alignment of BAA74848.1 (LBDS2) and 1QKN:A.

FIG. 8A: LigEye for 1QKN:A that illustrates the sites of interaction of Raloxifene (RAL600) with the Ligand Binding Domain of Rattus norvegicus Estrogen Receptor Beta, 1QKN:A.

FIG. 8B: iRasMol view of 1QKN:A, the Ligand Binding Domain of Rattus norvegicus Estrogen Receptor Beta.

### EXAMPLES

### Example 1: BAA74848.1 (LBDS2)

In order to initiate a search for novel, distantly related Nuclear Hormone Receptor Ligand Binding Domains, an archetypal family member is chosen, the Ligand Binding Domain of Rattus norvegicus Estrogen Receptor Beta. More specifically, the search is initiated using a structure from the Protein Data Bank (PDB) which is operated by the Research Collaboratory for Structural Bioinformatics.

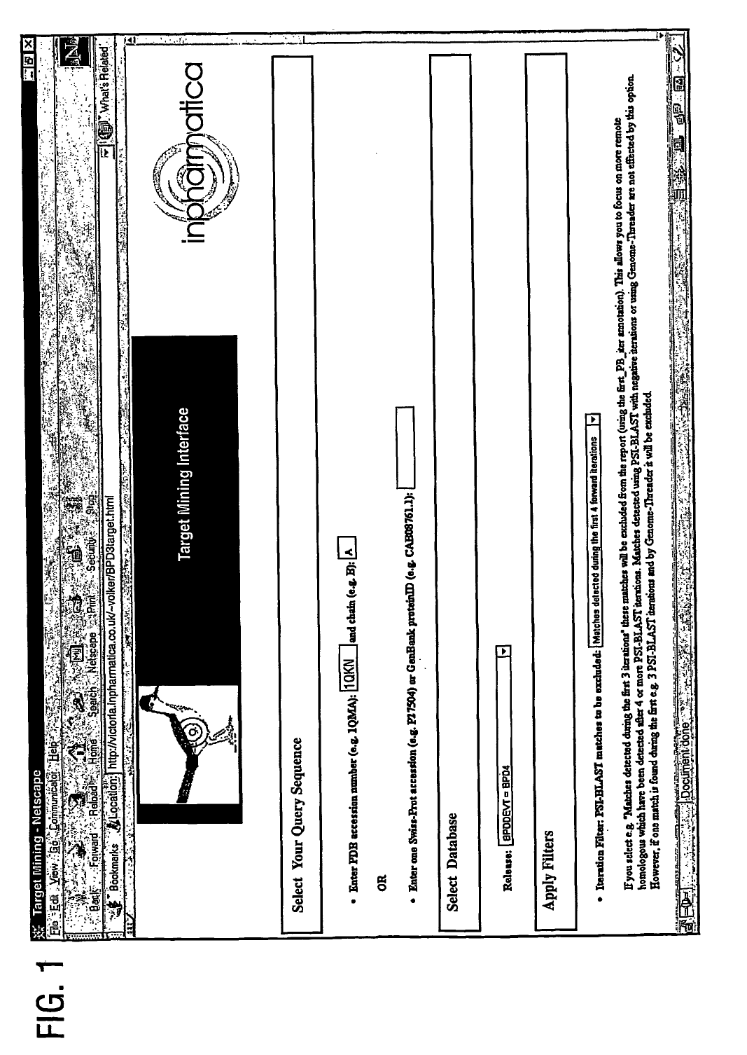

The structure chosen is the Ligand Binding Domain of Rattus norvegicus Estrogen Receptor Beta (PDB code 1QKN:A; see FIG. 1).

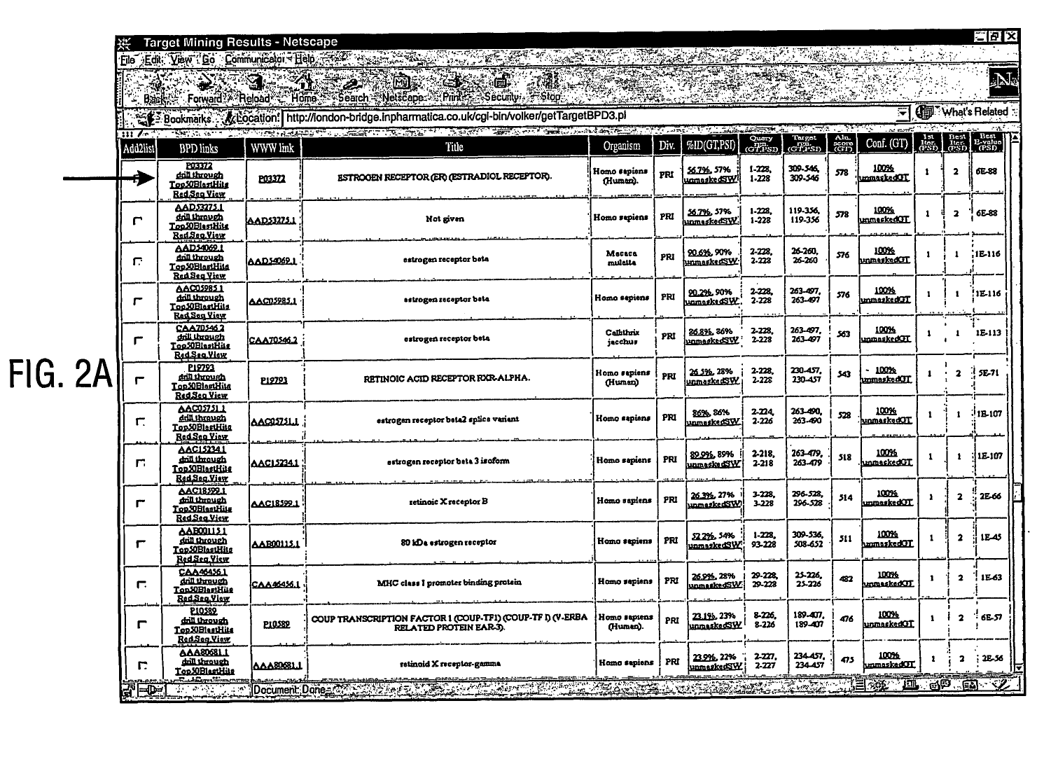

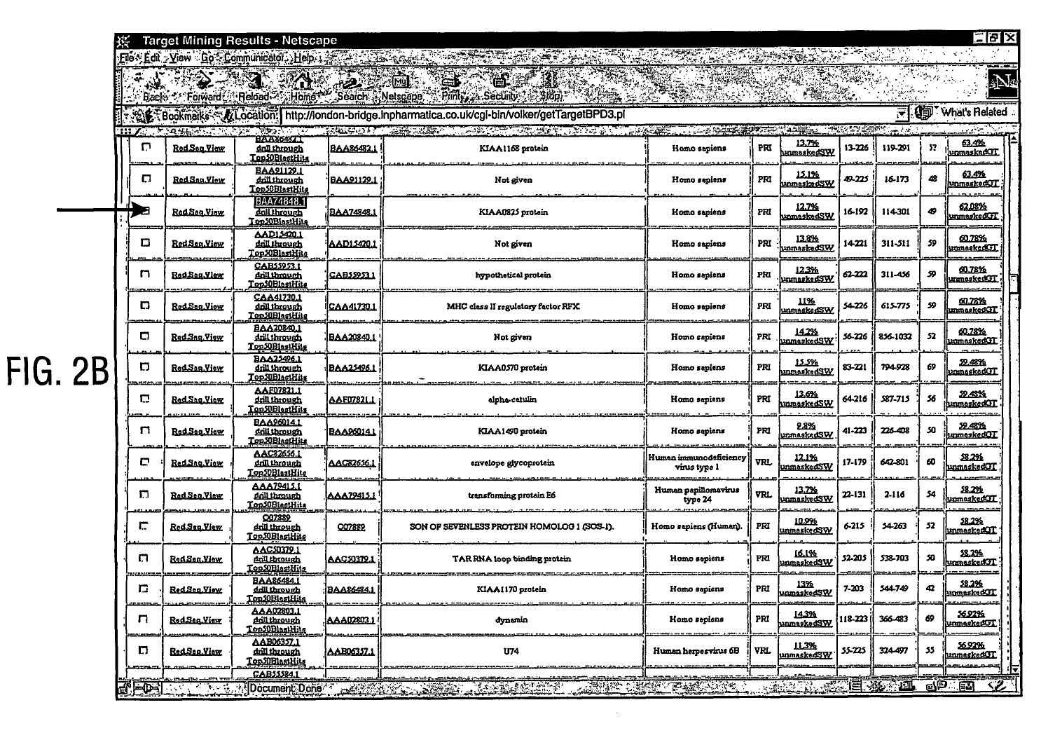

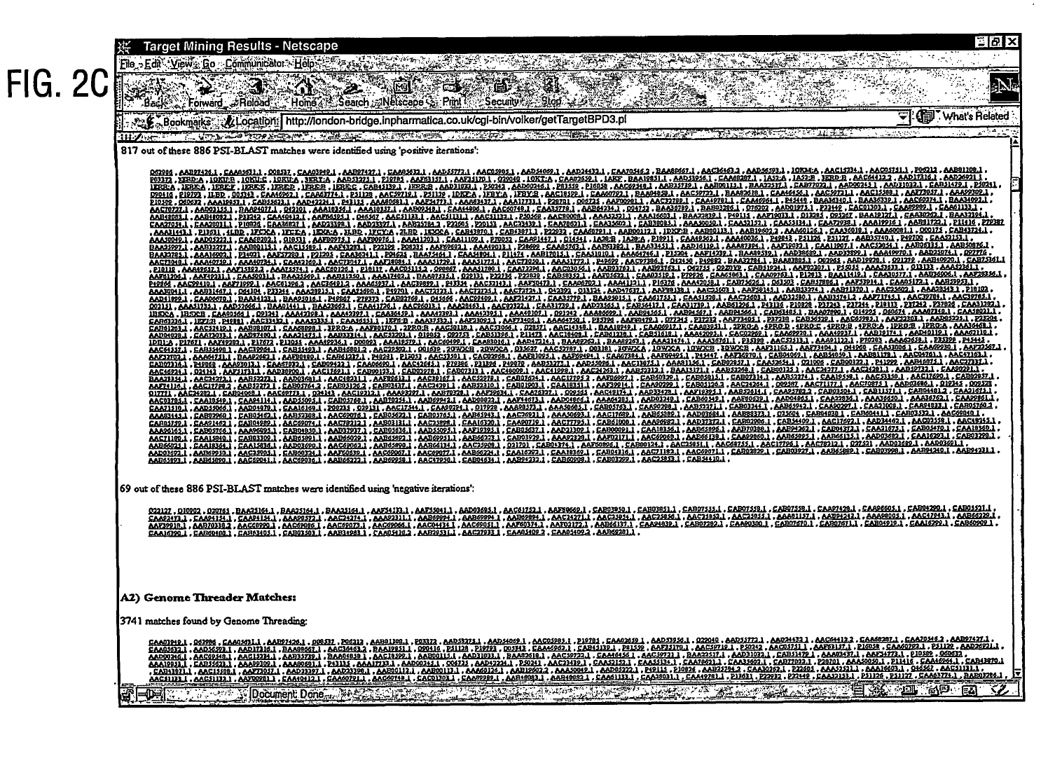

A search of the Biopendium (using the Target Mining Interface) for relatives of 1QKN:A takes place and returns 3741 Genome Threader results. The 3741 Genome Threader results include examples of typical Nuclear Hormone Receptor Ligand Binding Domains, such as that found between residues 309-546 of the Homo sapiens Estrogen Receptor (see arrow in FIG. 2A).

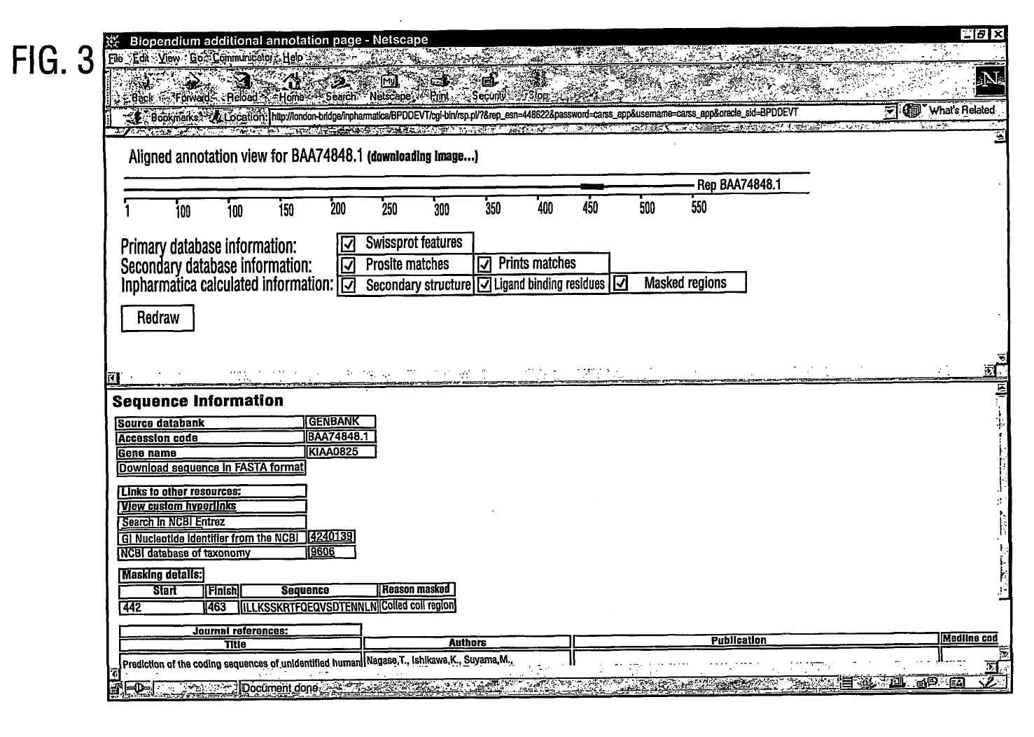

Among the proteins known to contain a Nuclear Hormone Receptor Ligand Binding Domain appears a protein which is not annotated as containing a Nuclear Hormone Receptor Ligand Binding Domain, BAA74848.1 (LBDS2; see arrow in FIG. 3B). The Inpharmatica Genome Threader has identified a region of the sequence BAA74848.1 (LBDS2), between residues 114-301, as having a structure similar to the Ligand Binding Domain of Rattus norvegicus Estrogen Receptor Beta. The possession of a structure similar to the Ligand Binding Domain of Rattus norvegicus Estrogen Receptor Beta suggests that residues 114-301 of BAA74848.1 (LBDS2) finction as a Nuclear Hormone Receptor Ligand Binding Domain. The Genome Threader identifies this with 62% confidence.

The search of the Biopendium (using the Target Mining Interface) for relatives of 1QKN:A also returns 817 Forward PSI-Blast results. Forward PSI-Blast (see FIG. 2C) is unable to identify this relationship; only the Inpharmatica Genome Threader is able to identify BAA74848.1 (LBDS2) as containing a Nuclear Hormone Receptor Ligand Binding Domain.

In order to assess what is known in the public domain databases about BAA74848.1 (LBDS2) the Redundant Sequence Display Page (FIG. 3) is viewed. There are no PROSITE or PRINTS hits which identify BAA74848.1 (LBDS2) as containing a Nuclear Hormone Receptor Ligand Binding Domain. PROSITE and PRINTS are databases that help to describe proteins of similar families. Returning no Nuclear Hormone Receptor Ligand Binding Domain hits from both databases means that BAA74848.1 (LBDS2) is unidentifiable as containing a Nuclear Hormone Receptor Ligand Binding Domain using PROSITE or PRINTS.

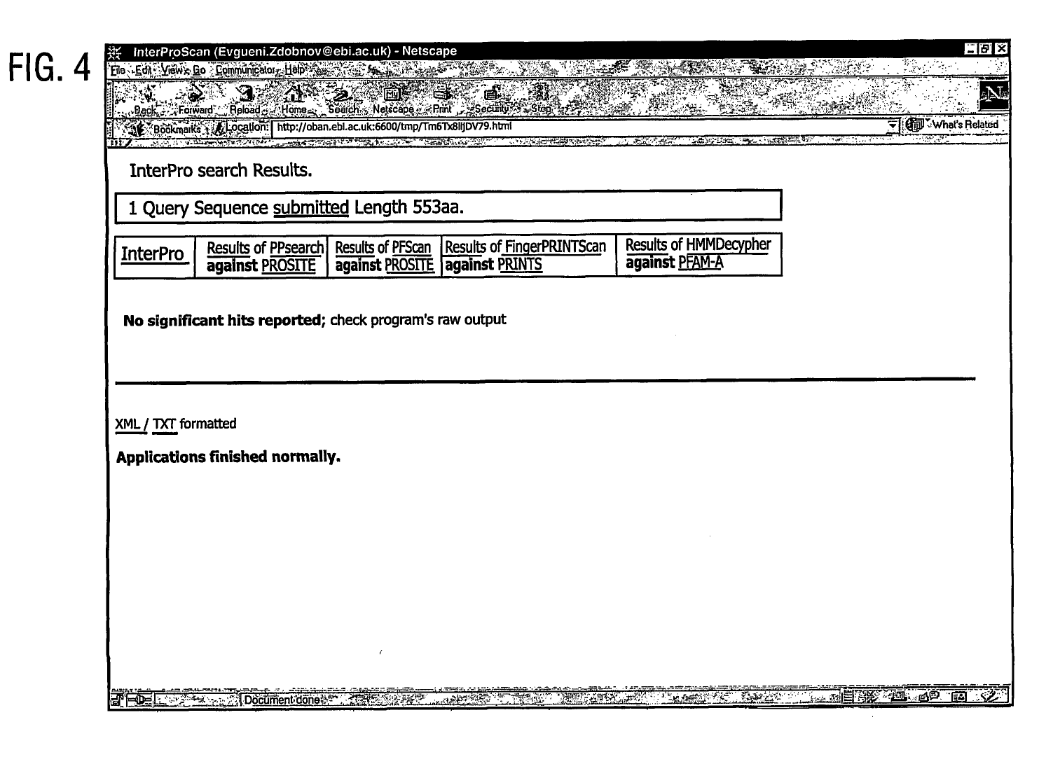

In order to identify if any other public domain annotation vehicle is able to annotate BAA74848.1 (LBDS2) as containing a Nuclear Hormone Receptor Ligand Binding Domain, the BAA74848.1 (LBDS2) protein sequence is searched against the PFAM database (Protein Family Database of Alignment and hidden Markov models) at the InterPro website (see FIG. 4). There are no PFAM-A matches annotating BAA74848.1 (LBDS2) as containing a Nuclear Hormone Receptor Ligand Binding Domain. Thus PFAM does not identify BAA74848.1 (LBDS2) as containing a Nuclear Hormone Receptor Ligand Binding Domain.

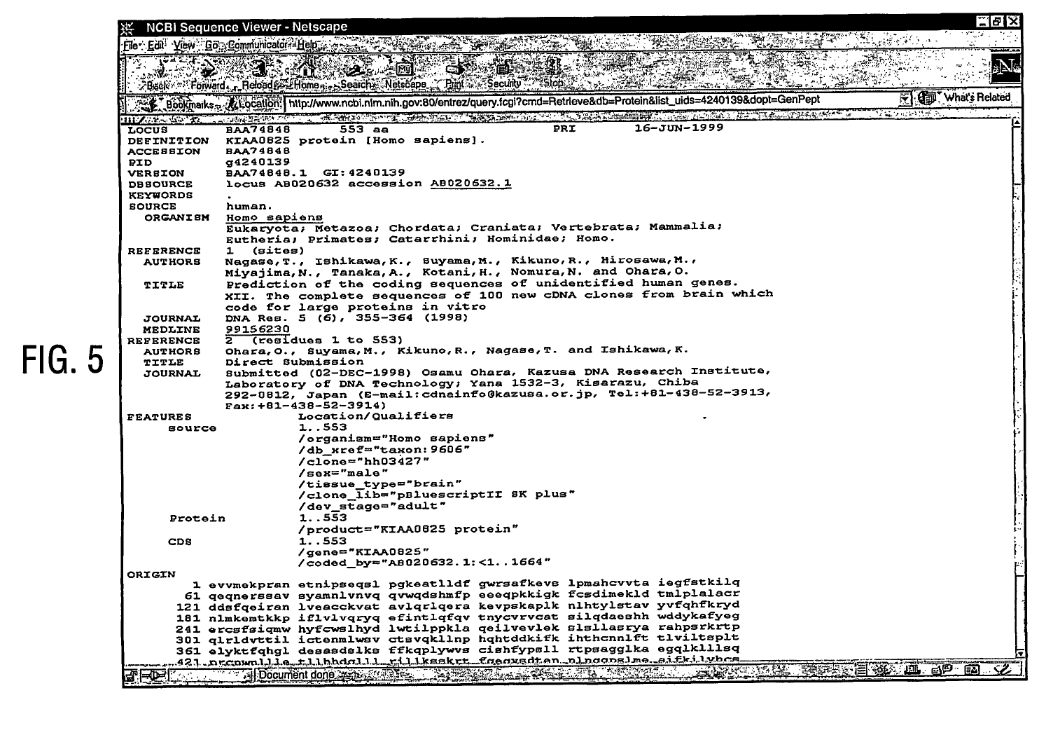

The National Center for Biotechnology Information (NCBI) Genebank protein database is then viewed to examine if there is any further information that is known in the public domain relating to BAA74848.1 (LBDS2). This is the US public domain database for protein and gene sequence deposition (FIG. 5). BAA74848.1 (LBDS2) is a Homo sapiens sequence, its Genebank protein ID is BAA74848.1 and it is 553 amino acids in length. BAA74848.1 (LBDS2) is called KIAA0825 protein. BAA74848.1 (LBDS2) was cloned by a group of scientists at the Kazusa DNA Research Institute, Laboratory of DNA Technology, Yana 1532-3, Kisarazu, Chiba, Japan. The public domain information for this gene does not annotate it as containing a Nuclear Hormone Receptor Ligand Binding Domain.

Therefore, it can be concluded that using all public domain annotation tools, BAA74848.1 (LBDS2) may not be annotated as containing a Nuclear Hormone Receptor Ligand Binding Domain. Only the Inpharmatica Genome Threader is able to annotate this protein as containing a Nuclear Hormone Receptor Ligand Binding Domain.

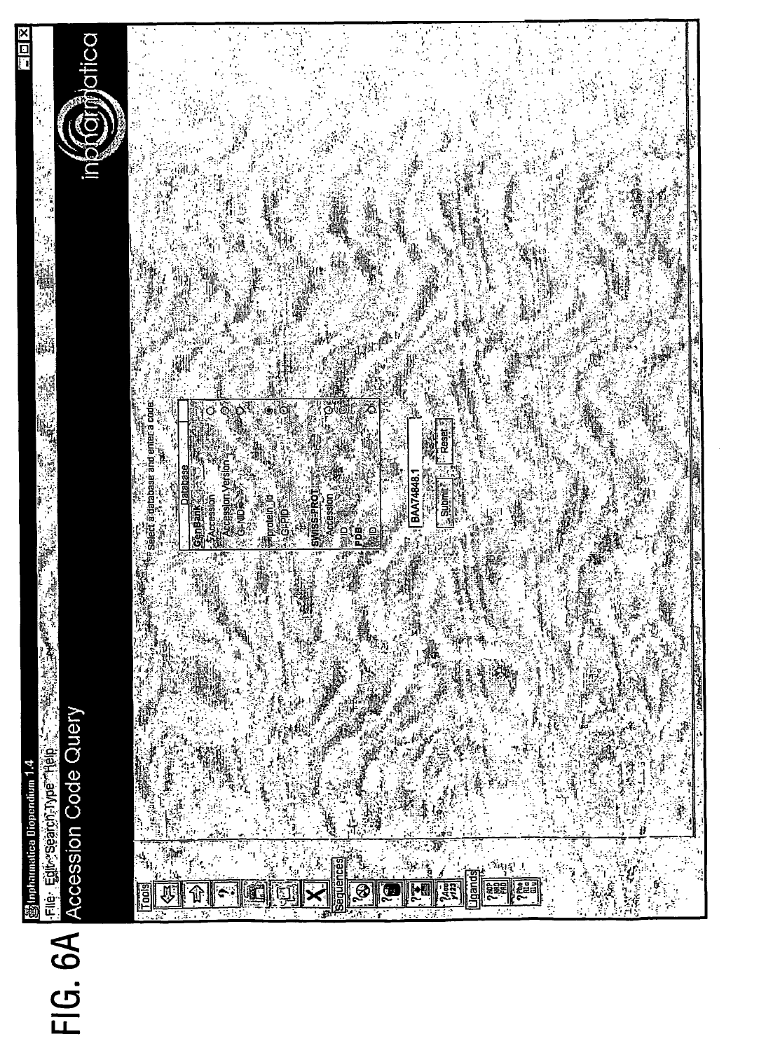

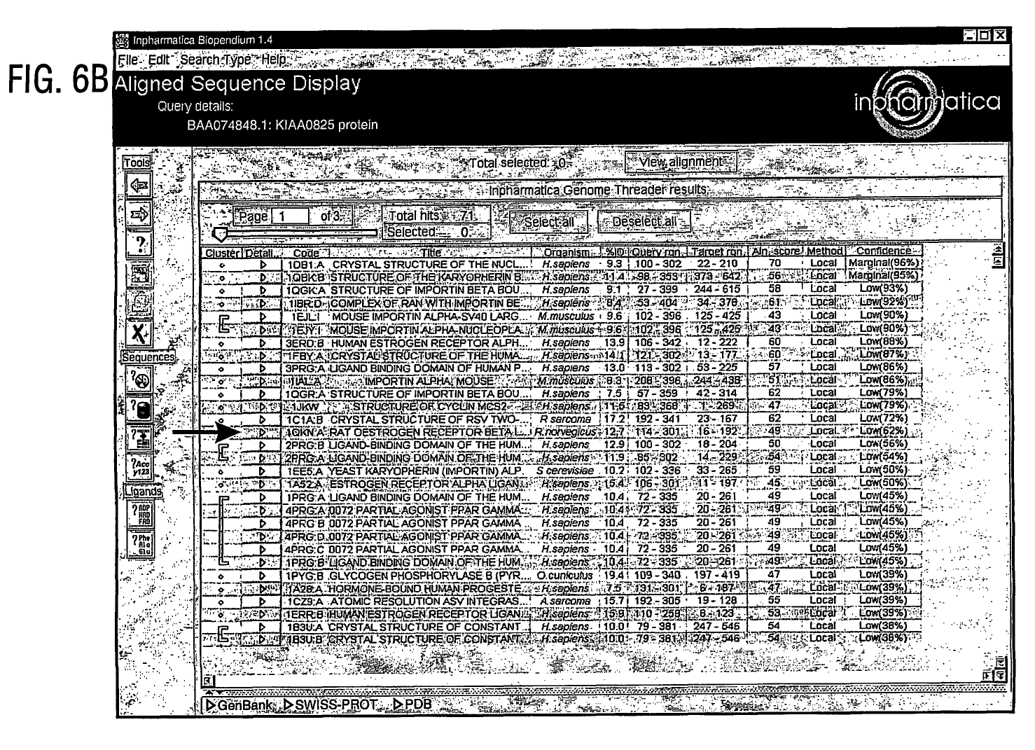

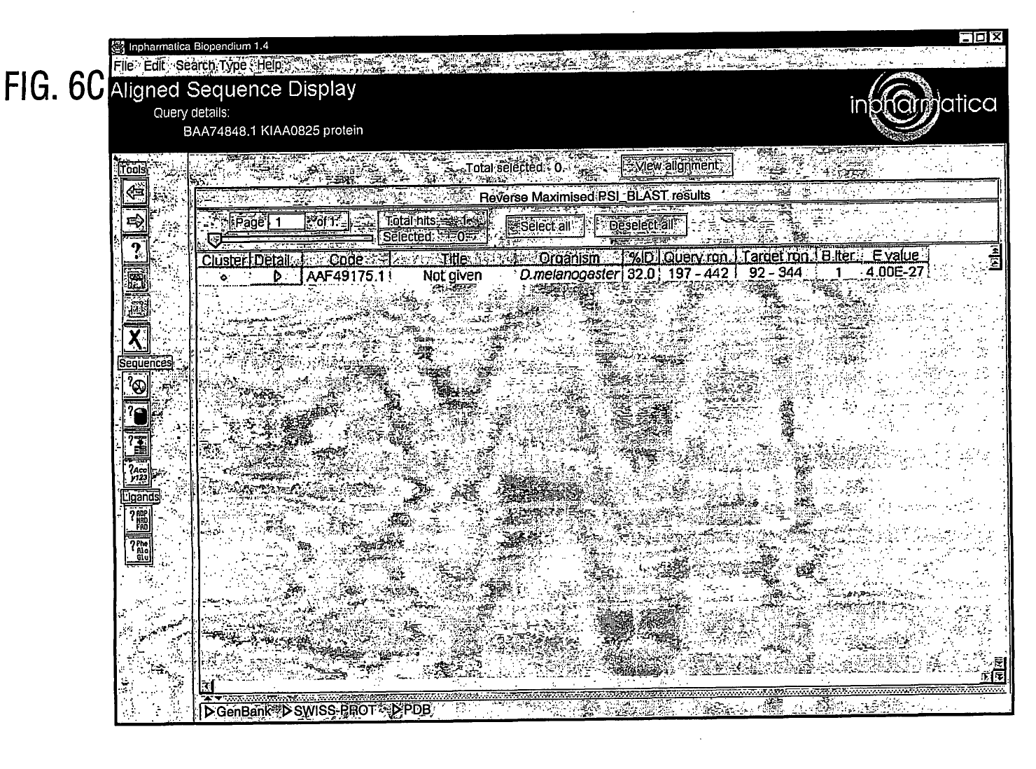

The reverse search is now carried out. BAA74848.1 (LBDS2) is now used as the query sequence in the Biopendium (see FIG. 6A). The Inpharmatica Genome Threader identifies residues 114-301 of BAA74848.1 (LBDS2) as having a structure that is the same as the Ligand Binding Domain of Rattus norvegicus Estrogen Receptor Beta with 62% confidence (see arrow in FIG. 6B). The Ligand Binding Domain of Rattus norvegicus Estrogen Receptor Beta (1QKN:A) was the original query sequence. Positive iterations of PSI-Blast do not return this result (FIG. 6C). It is only the Inpharmatica Genome Threader that is able to identify this relationship.

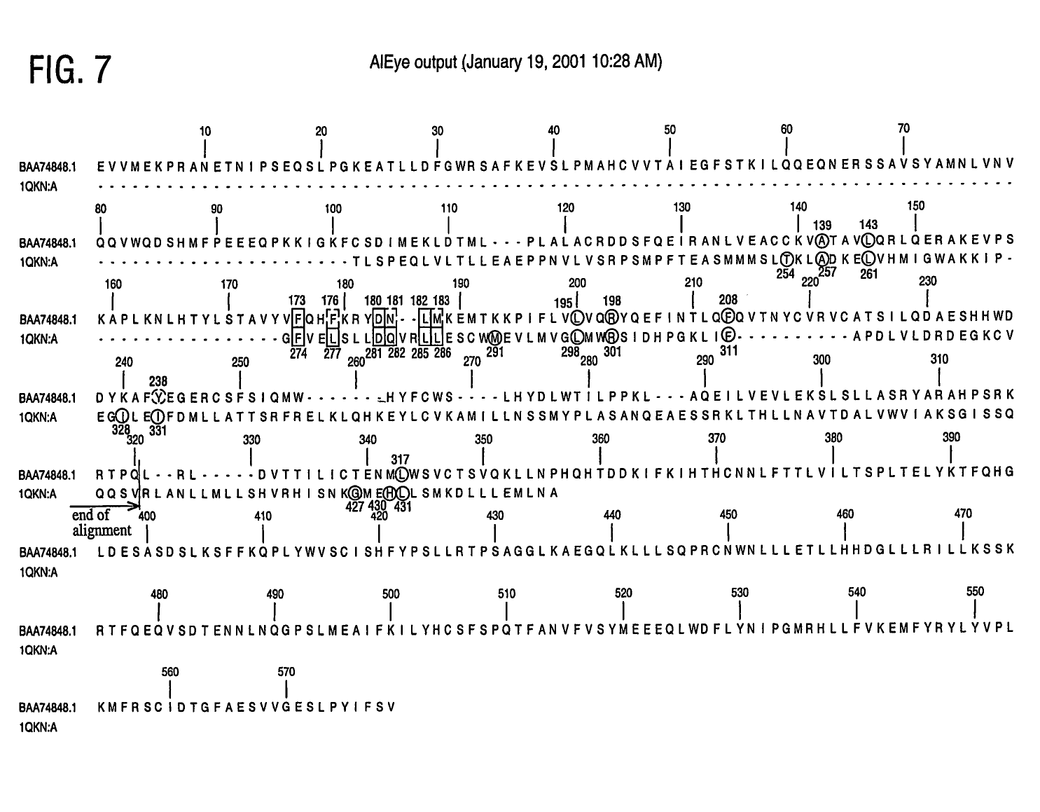

The sequence of the Rattus norvegicus Estrogen Receptor Beta Ligand Binding Domain, 1QKN:A is chosen against which to view the sequence alignment of BAA74848.1 (LBDS2). Viewing the AlEye alignment (FIG. 7) of the query protein against the protein identified as being of a similar structure helps to visualize the areas of homology.

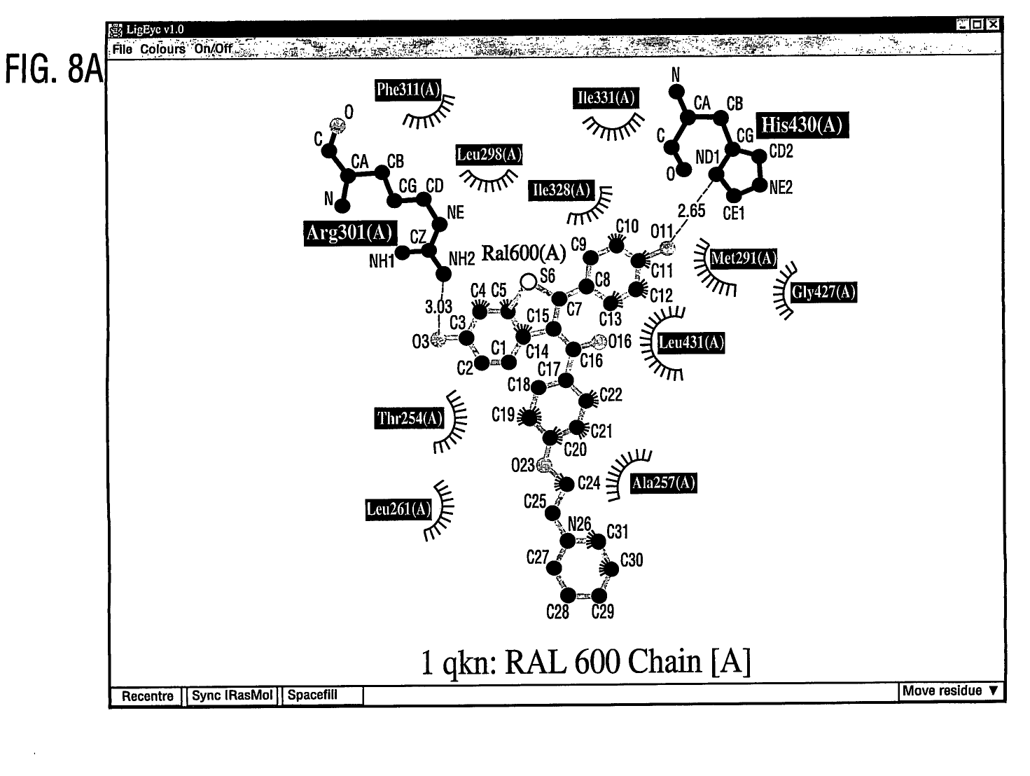

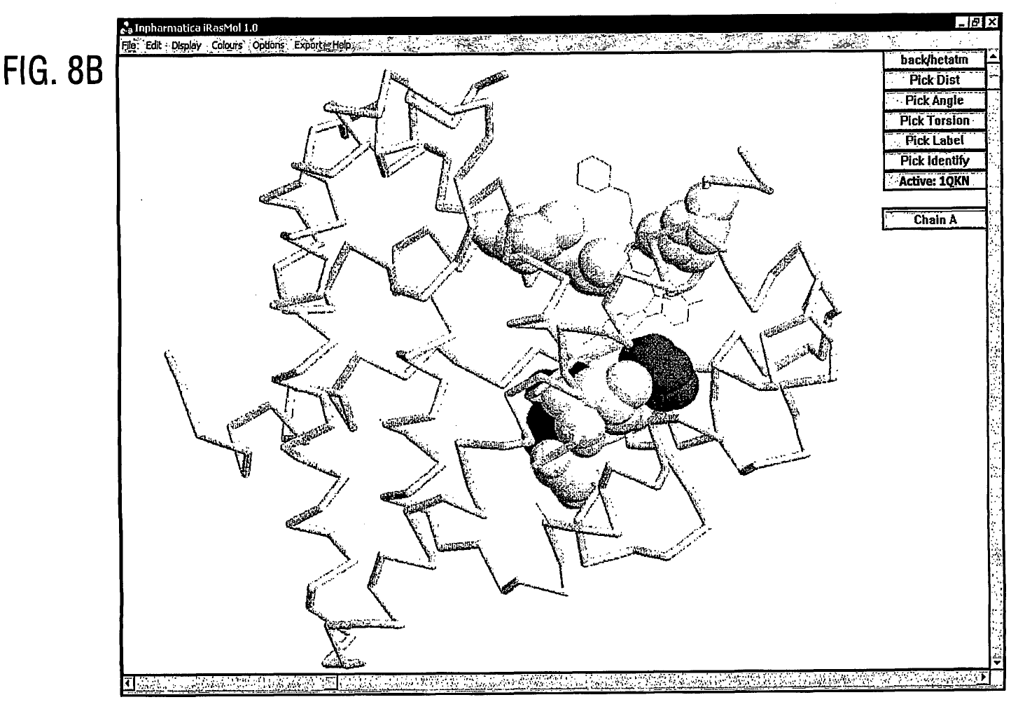

The Rattus norvegicus Estrogen Receptor Beta Ligand Binding Domain contains an “LBD motif” which has been found in all annotated Nuclear Hormone Receptor Ligand Binding Domains to date. The “LBD motif” is involved in recruiting Nuclear Hormone Receptor Co-Activators and Co-Repressors. The 6 residues; PHE274, LEU277, ASP281, GLN282, LEU285 and LEU286 constitute this motif in the Rattus norvegicus Estrogen Receptor Beta Ligand Binding Domain (see square boxes FIG. 8). 3 residues (PHE173, ASP180, LEU182) in BAA74848.1 (LBDS2) precisely match 3 (PHE274, ASP281, LEU285) out of the 6 “LBD motif” residues in the Rattus norvegicus Estrogen Receptor Beta Ligand Binding Domain. Furthermore PHE176, ASN181 and MET183 in BAA74848.1 (LBDS2) conservatively substitute for the remaining 3 residues (LEU277, GLN282 and LEU286 respectively) in the “LBD motif” of Rattus norvegicus Estrogen Receptor Beta Ligand Binding Domain. This indicates that BAA74848.1 (LBDS2) contains a Nuclear Hormone Receptor Ligand Binding Domain similar to The Rattus norvegicus Estrogen Receptor Beta Ligand Binding Domain.

In order to ensure that the protein identified is in fact a relative of the query sequence, the visualization programs “LigEye” (FIG. 8A) and “iRasmol” (FIG. 8B) are used. These visualization tools identify the active site of known protein structures by indicating the amino acids with which known small molecule inhibitors interact at the active site. These interactions are either through a direct hydrogen bond or through hydrophobic interactions. In this manner, one can see if the active site fold/structure is conserved between the identified homologue and the chosen protein of known structure. The LigEye view of the Rattus norvegicus Estrogen Receptor Beta Ligand Binding Domain reveals 12 residues which bind Raloxifene (circled in FIG. 7). However, only 9 (THR254, ALA257, LEU261, MET291, LEU298, ARG301, PHE311, ILE328 and ILE331) of these 12 residues lie within the Genome Threader alignment. Thus only these 9 residues can be used to consolidate the Genome Threader annotation of BAA74848.1 (LBDS2) as containing a Nuclear Hormone Receptor Ligand Binding Domain.

Of these 9 residues there are 7 hydrophobic residues which line the pocket of the Rattus norvegicus Estrogen Receptor Beta Ligand Binding Domain (ALA257, LEU261, MET291, LEU298, PHE311, ILE328 and ILE331). 4 of these hydrophobic residues (ALA257, LEU261, LEU298 and PHE311) are perfectly conserved in BAA74848.1 (LBDS2; ALA139, LEU143, LEU195 and PHE208; see FIG. 7). Furthermore, ILE331 of the Rattus norvegicus Estrogen Receptor Beta Ligand Binding Domain is replaced by the hydrophobic residue TYR238 in BAA74848.1 (LBDS2): (broken circle in FIG. 7). This conservation of hydrophobicity in 5 out of the 7 hydrophobic residues (within the region of Genome Threader alignment) which line the binding pocket indicates that BAA74848.1 (LBDS2) will bind a hydrophobic steroid-like ligand.

Typically, the binding pocket of Nuclear Hormone Receptor Ligand Binding Domains will contain a crucial arginine residue. The guanidinium group of this arginine functions to bind and stabilise a hydroxyl or carboxyl group present on the steroid/steroid-like ligand. For example, ARG394 of the Homo sapiens Estrogen Receptor Ligand Binding Domain binds and stabilises the phenolic hydroxyl group of the A ring of Estradiol (Brzozowski Nature 1997 vol.389:p753-758). LigEye shows that ARG301 of the Rattus norvegicus Estrogen Receptor Beta Ligand Binding Domain stabilises the O3 of Raloxifene. In BAA74848.1 (LBDS2) this ARG is perfectly conserved as ARG198. ARG198 in BAA74848.1 (LBDS2) could function to bind and stabilise a hydroxyl or carboxyl group present on a steroid-like ligand. This indicates that indeed as predicted by the Inpharmatica Genome Threader, BAA74848.1 (LBDS2) folds in a similar manner to the Rattus norvegicus Estrogen Receptor Beta Ligand Binding Domain and as such is identified as containing a Nuclear Hormone Receptor Ligand Binding Domain.

1. A polypeptide, which polypeptide:
(i) comprises or consists of the amino acid sequence as recited in SEQ ID NO:2;
(ii) is a fragment thereof having Nuclear Hormone Receptor Ligand Binding Domain activity or having an antigenic determinant in common with the polypeptide of (i); or
(iii) is a functional equivalent of (i) or (ii).

2. A polypeptide which is a fragment according to claim 1(ii), which includes the Nuclear Hormone Receptor Ligand Binding Domain region of the LBDS2 polypeptide, said Nuclear Hormone Receptor Ligand Binding Domain region being defined as including, residues 114 and 301 inclusive, of the amino acid sequence recited in SEQ ID NO:2, wherein said fragment possesses the “LBD motif” residues PHE173, PHE176, ASP180, ASN181, Leu182 and Met 183, or equivalent residues, and possesses Nuclear Hormone Receptor Ligand Binding Domain activity.

3. A polypeptide which is a functional equivalent according to claim 1(iii), is homologous to the amino acid sequence as recited in SEQ ID NO:2, possesses the catalytic residues PHE173, PHE176, ASP180, ASN181, LEU182 and MET 183, or equivalent residues, and has Nuclear Hormone Receptor Ligand Binding Domain activity.

4. A polypeptide according to claim 3, wherein said functional equivalent is homologous to the Nuclear Hormone Receptor Ligand Binding Domain region of the LBDS2 polypeptide.

5. A fragment or functional equivalent according to any one of claims 1-4, which has greater than 80% sequence identity with an amino acid sequence as recited in SEQ ID NO:2, or with a fragment thereof that possesses Nuclear Hormone Receptor Ligand Binding Domain activity, preferably greater than 85%, 90%, 95%, 98% or 99% sequence identity, as determined using BLAST version 2.1.3 using the default parameters specified by the NCBI (the National Center for Biotechnology Information; http://www.ncbi.nlm.nih.gov/) [Blosum 62 matrix; gap open penalty=11 and gap extension penalty=1].

6. A functional equivalent according to any one of claims 1-5, which exhibits significant structural homology with a polypeptide having the amino acid sequence given in any one of SEQ ID NO:2, or with a fragment thereof that possesses Nuclear Hormone Receptor Ligand Binding Domain activity.

7. A fragment as recited in claim 1, 2, or 5, having an antigenic determinant in common with the polypeptide of claim 1(i), which consists of 7 or more (for example, 8, 10, 12, 14, 16, 18, 20 or more) amino acid residues from the sequence of SEQ ID NO:2.

8. A purified nucleic acid molecule which encodes a polypeptide according to any one of the preceding claims.

9. A purified nucleic acid molecule according to claim 8, which has the nucleic acid sequence as recited in SEQ ID NO:1, or is a redundant equivalent or fragment thereof.

10. A fragment of a purified nucleic acid molecule according to claim 8 or claim 9, which comprises of nucleotides 342 to 905 inclusive of SEQ ID NO:1, or is a redundant equivalent thereof.

11. A purified nucleic acid molecule which hybridizes under high stringency conditions with a nucleic acid molecule according to any one of claims 8-10.

12. A vector comprising a nucleic acid molecule as recited in any one of claims 8-11.

13. A host cell transformed with a vector according to claim 12.

14. A ligand which binds specifically to, and which preferably inhibits the Nuclear Hormone Receptor Ligand Binding Domain activity of a polypeptide according to any one of claims 1-7.

15. A ligand according to claim 14, which is an antibody.

16. A compound that either increases or decreases the level of expression or activity of a polypeptide according to any one of claims 1-7.

17. A compound according to claim 16 that binds to a polypeptide according to any one of claims 1-7 without inducing any of the biological effects of the polypeptide.

18. A compound according to claim 16 or claim 17, which is a natural or modified substrate, ligand, enzyme, receptor or structural or functional mimetic.

19. A polypeptide according to any one of claims 1-7, a nucleic acid molecule according to any one of claims 8-11, a vector according to claim 12, a ligand according to claim 14 or 15, or a compound according to any one of claims 16-18, for use in therapy or diagnosis of disease.

20. A method of diagnosing a disease in a patient, comprising assessing the level of expression of a natural gene encoding a polypeptide according to any one of claims 1-7, or assessing the activity of a polypeptide according to any one of claims 1-7, in tissue from said patient and comparing said level of expression or activity to a control level, wherein a level that is different to said control level is indicative of disease.

21. A method according to claim 20 that is carried out in vitro.

22. A method according to claim 20 or claim 21, which comprises the steps of: (a) contacting a ligand according to claim 14 or claim 15 with a biological sample under conditions suitable for the formation of a ligand-polypeptide complex; and (b) detecting said complex.

23. A method according to claim 20 or claim 21, comprising the steps of:
a) contacting a sample of tissue from the patient with a nucleic acid probe under stringent conditions that allow the formation of a hybrid complex between a nucleic acid molecule according to any one of claims 8-11 and the probe;
b) contacting a control sample with said probe under the same conditions used in step a); and
c) detecting the presence of hybrid complexes in said samples;
wherein detection of levels of the hybrid complex in the patient sample that differ from levels of the hybrid complex in the control sample is indicative of disease.

24. A method according to claim 20 or claim 21, comprising:
a) contacting a sample of nucleic acid from tissue of the patient with a nucleic acid primer under stringent conditions that allow the formation of a hybrid complex between a nucleic acid molecule according to any one of claims 8-11 and the primer;
b) contacting a control sample with said primer under the same conditions used in step a); and
c) amplifying the sampled nucleic acid; and
d) detecting the level of amplified nucleic acid from both patient and control samples;
wherein detection of levels of the amplified nucleic acid in the patient sample that differ significantly from levels of the amplified nucleic acid in the control sample is indicative of disease.

25. A method according to claim 20 or claim 21 comprising:
a) obtaining a tissue sample from a patient being tested for disease;
b) isolating a nucleic acid molecule according to any one of claims 8-11 from said tissue sample; and
c) diagnosing the patient for disease by detecting the presence of a mutation which is associated with disease in the nucleic acid molecule as an indication of the disease.

26. The method of claim 25, further comprising amplifying the nucleic acid molecule to form an amplified product and detecting the presence or absence of a mutation in the amplified product.

27. The method of either claim 25 or 26, wherein the presence or absence of the mutation in the patient is detected by contacting said nucleic acid molecule with a nucleic acid probe that hybridises to said nucleic acid molecule under stringent conditions to form a hybrid double-stranded molecule, the hybrid double-stranded molecule having an unhybridised portion of the nucleic acid probe strand at any portion corresponding to a mutation associated with disease; and
detecting the presence or absence of an unhybridised portion of the probe strand as an indication of the presence or absence of a disease-associated mutation.

28. A method according to any one of claims 20-27, wherein said disease is cell proliferative disorders, including neoplasm, cancer and myeloproliferative disorders and angiogenesis, metabolic disorders, including obesity, osteoporosis, non-insulin dependent diabetes, lipid metabolism disorder, hyperthyroidism, hyperparathyroidism, hypercalcemia, hypercholestrolemia, hyperlipidemia, cardiovascular disorders including, hypertension, athersclerosis, hyperinsulinemia, heart arrhythmia, renal disorders, including glomerulonephritis, renovascular hypertension, skin disorders including dermatological disease, psoriasis, acne, aging, eczema, wound healing, inflammation, including inflammatory bowel disease, respiratory tract inflammation, emphysema, asthma, immune disorder, autoimmune disease, multiple sclerosis, nervous system disorders, including anxiety, depression, neurodegenerative disease, Alzheimer's disease Parkinson's disease, brain injury, stroke and pain, infection, including virus infection, and other conditions in which nuclear hormones are implicated.

29. Use of a polypeptide according to any one of claims 1-7 as a Nuclear Hormone Receptor Ligand Binding Domain.

30. Use of a nucleic acid molecule according to any one of claims 8-11 to express a protein that possesses Nuclear Hormone Receptor Ligand Binding Domain activity.

31. A method for effecting cell-cell adhesion, utilising a polypeptide according to any one of claims 1-7.

32. A pharmaceutical composition comprising a polypeptide according to any one of claims 1-7, a nucleic acid molecule according to any one of claims 8-11, a vector according to claim 12, a ligand according to claim 14 or 15, or a compound according to any one of claims 16-18.

33. A vaccine composition comprising a polypeptide according to any one of claims 1-7 or a nucleic acid molecule according to any one of claims 8-11.

34. A polypeptide according to any one of claims 1-7, a nucleic acid molecule according to any one of claims 8-11, a vector according to claim 12, a ligand according to claim 14 or 15, a compound according to any one of claims 16-18, or a pharmaceutical composition according to claim 32 for use in the manufacture of a medicament for the treatment of cell proliferative disorders, including neoplasm, cancer and myeloproliferative disorders and angiogenesis, metabolic disorders, including obesity, osteoporosis, non-insulin dependent diabetes, lipid metabolism disorder, hyperthyroidism, hyperparathyroidism, hypercalcemia, hypercholestrolemia, hyperlipidemia, cardiovascular disorders including, hypertension, athersclerosis, hyperinsulinemia, heart arrhythmia, renal disorders, including glomerulonephritis, renovascular hypertension, skin disorders including dermatological disease, psoriasis, acne, aging, eczema, wound healing, inflammation, including inflammatory bowel disease, respiratory tract inflammation, emphysema, asthma, immune disorder, autoimmune disease, multiple sclerosis, nervous system disorders, including anxiety, depression, neurodegenerative disease, Alzheimer's disease Parkinson's disease, brain injury, stroke and pain, infection, including virus infection, and other conditions in which nuclear hormones are implicated.

35. A method of treating a disease in a patient, comprising administering to the patient a polypeptide according to any one of claims 1-7, a nucleic acid molecule according to any one of claims 8-11, a vector according to claim 12, a ligand according to claim 14 or 15, a compound according to any one of claims 16-18, or a pharmaceutical composition according to claim 32.

36. A method according to claim 35, wherein, for diseases in which the expression of the natural gene or the activity of the polypeptide is lower in a diseased patient when compared to the level of expression or activity in a healthy patient, the polypeptide, nucleic acid molecule, vector, ligand, compound or composition administered to the patient is an agonist.

37. A method according to claim 35, wherein, for diseases in which the expression of the natural gene or activity of the polypeptide is higher in a diseased patient when compared to the level of expression or activity in a healthy patient, the polypeptide, nucleic acid molecule, vector, ligand, compound or composition administered to the patient is an antagonist.

38. A method of monitoring the therapeutic treatment of disease in a patient, comprising monitoring over a period of time the level of expression or activity of a polypeptide according to any one of claims 1-7, or the level of expression of a nucleic acid molecule according to any one of claims 8-11 in tissue from said patient, wherein altering said level of expression or activity over the period of time towards a control level is indicative of regression of said disease.

39. A method for the identification of a compound that is effective in the treatment and/or diagnosis of disease, comprising contacting a polypeptide according to any one of claims 1-7, a nucleic acid molecule according to any one of claims 8-11, or a host cell according to claim 13 with one or more compounds suspected of possessing binding affinity for said polypeptide or nucleic acid molecule, and selecting a compound that binds specifically to said nucleic acid molecule or polypeptide.

40. A kit useful for diagnosing disease comprising a first container containing a nucleic acid probe that hybridises under stringent conditions with a nucleic acid molecule according to any one of claims 8-11; a second container containing primers useful for amplifying said nucleic acid molecule; and instructions for using the probe and primers for facilitating the diagnosis of disease.

41. The kit of claim 40, further comprising a third container holding an agent for digesting unhybridised RNA.

42. A kit comprising an array of nucleic acid molecules, at least one of which is a nucleic acid molecule according to any one of claims 8-11.

43. A kit comprising one or more antibodies that bind to a polypeptide as recited in any one of claims 1-7; and a reagent useful for the detection of a binding reaction between said antibody and said polypeptide.

44. A transgenic or knockout non-human animal that has been transformed to express higher, lower or absent levels of a polypeptide according to any one of claims 1-7.

45. A method for screening for a compound effective to treat disease, by contacting a non-human transgenic animal according to claim 44 with a candidate compound and determining the effect of the compound on the disease of the animal.
